(19) United States
(12) Patent Application Publication
(10) Pub. No.: US 2024/0345097 A1
(43) Pub. Date: Oct. 17, 2024

(54) E3 UBIQUITIN LIGASE (UBE3A) PROTEIN TARGETS

(71) Applicant: Hoffmann-La Roche Inc., Little Falls, NJ (US)

(72) Inventors: Ravi JAGASIA, Basel (CH); Thomas KREMER, Grenzach-Wyhlen (DE); Sonja MEIER, Basel (CH); Nikhil Janak PANDYA, San Mateo, CA (US); Stefka Grigorova TYANOVA, Basel (CH)

(73) Assignee: Hoffmann-La Roche Inc., Little Falls, NJ (US)

(21) Appl. No.: 18/441,878

(22) Filed: Feb. 14, 2024

Related U.S. Application Data
(63) Continuation of application No. PCT/EP2022/072744, filed on Aug. 15, 2022.

(30) Foreign Application Priority Data
Aug. 16, 2021 (EP) 21191417.1

(51) Int. Cl. G01N 33/68; G01N 33/50

(52) U.S. Cl. CPC G01N 33/6848; G01N 33/5023

(57) ABSTRACT

The invention relates to UBE3A protein targets and their usage as target engagement biomarkers for compounds that modulate ube3a expression.

### CROSS-REFERENCE TO RELATED APPLICATIONS

This application is a continuation of International Application No. PCT/EP2022/072744 having an international filing date of Aug. 15, 2022, which claims benefit of priority to European Patent Application No. 21191417.1, filed Aug. 16, 2021, each of which is incorporated herein by reference in its entirety.

### SEQUENCE LISTING

This application contains a Sequence Listing which has been submitted electronically in XML format and is hereby incorporated by reference in its entirety. Said XML copy, created on Feb. 2, 2024, is named P36306-US_Sequence_Listing.xml and is 13,473 bytes in size.

### FIELD OF THE INVENTION

The present invention provides novel biomarkers whose protein expression levels are modulated when ubiquitin-protein ligase E3A (UBE3A) protein levels are increased or decreased and their use in drug development.

### BACKGROUND OF INVENTION

Angelman syndrome is characterized by severe intellectual and developmental disability, sleep disturbance, seizures, jerky movements, EEG abnormalities, frequent laughter or smiling, and profound language impairments. Angelman syndrome is neuro-genetic disorder caused by deletion or inactivation of the UBE3A genes and thus protein on the maternally inherited chromosome 15q11.2. Conversely, Dup15q Syndrome is a clinically identifiable syndrome which results from duplications of chromosome 15q11-13.1. In Dup15q Syndrome there is an overexpression of UBE3A. In Angelman syndrome (AS) the neuronal loss of E3 Ubiquitin ligase UBE3A leads to a plethora of severe neurological disabilities.

Although neuronal loss of UBE3 A causes AS, there is a paucity of knowledge of downstream molecular and cellular dysfunction. Identification of relevant UBE3A substrates, will lead to a better understanding of the role of Ube3a function in health and disease, and support both drug and biomarker discovery to monitor UBE3A function.

### SUMMARY OF THE INVENTION

The present invention relates to novel biomarkers whose protein expression is modulated when ubiquitin-protein ligase E3A (UBE3A) protein levels are increased or decreased and furthermore some are forming a protein complex with UBE3A. These include proteins TKT, DZANK1, ACYP1, UBLCP1, YARS, WARS, SOD2 and PSME3. The present invention further relates to pharmaceutical biomarkers and methods the detection of UBE3A activity based on these proteins for pharmaceutical treatment for diseases targeting UBE3A including Angelman syndrome, 15qdup syndrome and other Autism Spectrum Disorders.

### DETAILED DESCRIPTION OF THE INVENTION

In a first aspect, the present invention provides a method for measuring UBE3A protein expression modulation in a tissue sample comprising the steps:
a) providing a tissue sample of an animal or cell culture which has been treated with a UBE3A modulator,
b) measuring a protein expression level in the sample of step a) of at least one protein selected from the group consisting of: TKT, DZANK1, ACYP1, UBLCP1, YARS, WARS, SOD2 and PSME3.
c) comparing the protein expression level of the at least one protein measured in step b) to the protein expression level of the at least one protein in a control sample, wherein a modulated protein expression level of the at least one protein measured in step b) compared to the protein expression level of the at least one protein in the control sample is indicative for UBE3A protein expression modulation.

In a second aspect, the present invention relates to a method for measuring UBE3A protein expression induction in a tissue sample comprising the steps:
a) providing a tissue sample of an animal or cell culture which has been treated with a UBE3A inducer,
b) measuring a protein expression level in the sample of step a) of at least one protein selected from the group consisting of: TKT, DZANK1, ACYP1, UBLCP1, YARS, WARS, SOD2 and PSME3.
c) comparing the protein expression level of the at least one protein measured in step b) to the protein expression level of the at least one protein in a control, wherein a decreased protein expression level of the at least one protein measured in step b) compared to the protein expression level of the at least one protein in the control is indicative for UBE3A protein expression induction.

In a further aspect, the present invention relates to a method for determining UBE3A target engagement of an UBE3A modulator comprising the steps:
a) providing a tissue sample of an animal or cell culture which has been treated with a UBE3A modulator,
b) measuring a protein expression level in the sample of step a) of at least one protein selected from the group consisting of: TKT, DZANK1, ACYP1, UBLCP1, YARS, WARS, SOD2 and PSME3.
c) comparing the protein expression level of the at least one protein measured in step b) to the protein expression level of the at least one protein in a control, wherein a modulated protein expression level of the at least one protein measured in step b) compared to the protein expression level of the at least one protein in the control is indicative for UBE3A target engagement of the UBE3A modulator.

In a further aspect the present invention relates to a screening method for the identification of UBE3A protein expression modulators comprising the steps:
a) providing a tissue sample of an animal or cell culture which has been treated with a test compound,
b) measuring a protein expression level in the sample of step a) of at least one protein selected from the group consisting of: TKT, DZANK1, ACYP1, UBLCP1, YARS, WARS, SOD2 and PSME3.
c) comparing the protein expression level of the at least one protein measured in step b) to the protein expression level of the at least one protein in a control, wherein a modulated protein expression level of the at least one protein measured in step b) compared to the protein expression level of the at least one protein in the control is indicative for a UBE3A protein expression modulator.

In a particular embodiment, the tissue sample is a blood sample, a plasma sample or a CSF sample.

In a particular embodiment, the protein expression level is measured using Western blotting, Mass spectrometry (MS), Liquid chromatography-mass spectrometry (LC-MS) or immunoassays.

In a particular embodiment, the UBE3A modulator is an antisense oligonucleotide, in particular a LNA antisense oligonucleotide.

In a particular embodiment, the UBE3A modulator is an UBE3A protein expression level inducer for the treatment of Autism Spectrum Disorder, Angelman Syndrome or 15qdup syndrome.

In a particular embodiment, the protein in step b) is selected from the group consisting of TKT, DZANK1, UBLCP1 and PSME3 and the expression level of these proteins inversely correlates to the UBE3A expression level.

In a particular embodiment, the protein in step b) is selected from the group consisting of ACYP1, YARS, WARS and SOD2 and the expression level of these proteins directly correlates to the expression level of UBE3A protein expression level.

In a further aspect, the present invention relates to a use of a protein selected from the group consisting of TKT, DZANK1, ACYP1, UBLCP1, YARS, WARS, SOD2 and PSME3 as biomarker for UBE3A protein expression level modulation.

In a particular embodiment of the use of the present invention, the UBE3A modulation is due to a UBE3A protein expression level inducer.

In a particular embodiment of the use of the present invention, the biomarker protein is selected from the group consisting of: TKT, DZANK1, UBLCP1 and PSME3 and the protein expression level of these biomarker proteins inversely correlates to the UBE3A protein expression level.

In a particular embodiment of the use of the present invention, the biomarker protein is selected from the group consisting of: ACYP1, YARS, WARS and SOD2 and the protein expression level of these biomarker proteins directly correlates to the UBE3A protein expression level.

In a particular embodiment of the use of the present invention, the present invention provides a method for determining UBE3A target engagement of an UBE3A protein expression level modulator.

In a particular embodiment of the use of the present invention, the UBE3A protein expression level modulator is an antisense oligonucleotide, in particular a LNA antisense oligonucleotide.

In a particular embodiment of the use of the present invention, the UBE3A protein expression level modulator is an UBE3A protein expression level inducer for the treatment of Autism Spectrum Disorder, Angelman Syndrome or 15qdup syndrome.

### Definitions

The term “protein,” as used herein, refers to any native protein from any vertebrate source, including mammals such as primates (e.g. humans) and rodents (e.g., mice and rats), unless otherwise indicated. The term encompasses “full-length,” unprocessed proteins as well as any form of protein which results from processing in the cell as well as peptides derived from the native protein. The term also encompasses naturally occurring variants e.g., splice variants or allelic variants. The amino acid sequences shown in Table 2 are exemplary amino acid sequences of the biomarker proteins of the present invention.

In the present invention, an UBE3A protein expression level modulator refers to a molecule capable of reducing or enhancing the protein expression level of UBE3A. A modulator capable of reducing the protein expression level of UBE3A is referred to as UBE3A inhibitor and a modulator capable of enhancing the protein expression level of UBE3A is referred to as UBE3A enhancer. An UBE3A modulator may be an mRNA interfering RNA molecule. In another embodiment, the UBE3A modulator is a double-stranded RNA (dsRNA), for example, a short interfering RNA (siRNA) or a short hairpin RNA (shRNA). The double-stranded RNA may be any type of RNA, including but not limited to mRNA, snRNA, microRNA, and tRNA. RNA interference (RNAi) is particularly useful for specifically inhibiting the production of specific RNA and/or proteins. The design and production of dsRNA molecules suitable for the present invention are within the skill of those skilled in the art, particularly with reference to WO 99/32619, WO 99/53050, WO 99/49029 and WO 01/34815. Preferably siRNA molecule comprises a nucleotide sequence having about 19 to 23 contiguous nucleotides identical to the target mRNA. The term “shRNA” refers to a siRNA molecule in which fewer than about 50 nucleotides pair with the complementary sequence on the same RNA molecule, which sequence and complementary sequence are separated by an unpaired region of at least about 4 to 15 nucleotides (forming a single-chain loop on the stem structure produced by the two base-complementary regions). There are well-established siRNA design criteria (see, for example, Elbashire et al., 2001).

The UBE3A modulator can be an antisense oligonucleotide which is capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides are single stranded. It is understood that single stranded oligonucleotides can form hairpins or intermolecular duplex structures (duplex between two molecules of the same oligonucleotide), as long as the degree of intra or inter self-complementarity is less than 50% across of the full length of the oligonucleotide. The UBE3A modulator can be a gene therapy which establishes the expression of a functional UBE3A protein in a patient in need thereof.

The term “control sample” refers to a sample which has not been treated with a UBE3A modulator. For example, the control sample is a sample of a cell culture which has not been treated with a UBE3A modulator or the cell culture has been treated with a compound which is not a UBE3A modulator (negative control).

The expression level of the UBE3 A marker proteins TKT, DZANK1, UBLCP1 and PSME3 inversely correlates with the expression level of UBE3A protein i.e. a low level of UBE3A protein level correlates with a high expression level of these marker proteins and an increase in UBE3A protein level correlates with a decrease of these marker protein expression levels.

The expression level of the UBE3A marker proteins ACYP1, YARS, WARS and SOD2 directly correlates with the expression level of UBE3A protein i.e. a low level of UBE3A protein expression correlates with a low expression level of these marker proteins and an increase in UBE3A protein level correlates with an increase of these marker protein expression levels.

### SHORT DESCRIPTION OF THE FIGURES

FIGS. 1A-1E: AS mice exhibit proteomic alterations at birth, which exacerbate into adolescence and adulthood.

FIG. 1A: Schematic representation of experimental design. Control and AS mice were sacrificed at P1, P21, and P56. Pooled cortical tissue of control and AS animals was used to generate a sample-specific spectral library for data-independent acquisition (DIA) mass spectrometry. Individual samples were run in DIA mode and data analyzed using the sample specific library. Protein expression data was subjected to statistical and pathway enrichment analysis.

FIG. 1B: UBE3A raw protein intensity plot of cortices of control and AS mice at P1, P21, and P56 plotted as percentage of P1 control protein levels (mean±S.E.M. n=5-6).

FIG. 1C: Partial least square-discriminant analysis (PLS-DA) performed on the total proteome of control and AS mouse cortices resolved according to age (T1; P1, P21, and P56) and genotype (T3; control and AS).

FIG. 1D: Pathway enrichment plot depicting normalized enrichment scores using 1D annotation function using GO: Cellular component genesets in AS vs. control mice.

FIG. 1E: Average Z-score heatmap per time point of significantly altered (p value<0.05) proteins in AS vs. control mice. Clusters are defined using Euclidean distance based on the UPGMA method.

FIGS. 2A-2G: Pathway alterations in amino-acyl tRNA synthetases, proteasome and synapse are developmentally regulated.

FIG. 2A: Average Z scored heatmap per time point for amino-acyl tRNA synthetases multienzyme complex and amino-acyl tRNA synthetases. Clusters are defined using Euclidian distance based on the UPGMA method.

FIG. 2B: Time course expression of select proteins from the amino-acyl tRNA synthetases pathway depicting up-regulated set (Aimp1, Mars) and down-regulated set (Yars, Wars) in AS vs controls. Values represent Z scored values. Error bars: s.e.m.

FIG. 2C: Average Z scored heatmap per time point for proteasome complex based on their sub-unit classification. Clusters are defined using Euclidian distance based on the UPGMA method.

FIG. 2D: Time course expression of select proteins pathway depicting up-regulated sets of proteins from the 20S core proteasome subunits (Psma5, Psmb1, Psmb2), 19S proteasome regulatory subunit (Psmc3, Psmc4 and Psmd11) and proteasome interacting proteins (Ublcp1, Uchl5 and Uspl4) in AS vs controls. Values represent Z scored values. Error bars: s.e.m.

FIG. 2E: Average Z scored heatmap per time point for proteins belonging to the term synapse that were significantly altered (p value<0.05) at time point P56. Clusters are defined using euclidean distance based on the UPGMA method. Clustering indicates a split between proteins that are up-regulated in AS (red cluster) and those that are downregulated in AS (blue cluster).

FIG. 2F and FIG. 2G: Sunburst visualization for proteins that are up-regulated in AS (FIG. 2F) or down regulated in AS (FIG. 2G). The genes are annotated against SynGO CC (SynGO). Colors in the sunburst plot represent enrichment Q value scores of the UP (red) or DOWN (blue) set versus the entire measurable proteome (7126 proteins) as background. Proteins belonging to the furthest edge from the central synaptic term are labeled.

FIGS. 3A-3K: Adult AS rats recapitulate the proteomic alterations observed in AS mice across different brain regions.

FIG. 3A: Schematic representation of experimental design. Control and AS rats were sacrificed at P84. Pooled tissue of cerebellum (CB), cortex (CX), and hippocampus (HC) of control and AS animals was used to generate a sample-specific spectral library in DDA (data dependent acquisition) mode. Individual samples were further analyzed using data-independent acquisition (DIA) mass spectrometry.

FIG. 3B: UBE3A raw protein intensity plot of cerebellum (CB), cortex (CX), and hippocampus (HC) of control and AS rats plotted as percentage of CB control protein levels (mean±S.E.M.).

FIG. 3C: Partial least square-discriminant analysis (PLS-DA) performed on the total proteome of control and AS rats resolved according to brain region (T1 and T2; cerebellum, cortex, hippocampus) and genotype (T3; control and AS), and projected in 3D space.

FIG. 3D: Venn diagram of statistically significant (adj. p value<0.05) proteins altered in each brain region in AS rats.

FIG. 3E: Heatmap of proteins that pass statistical significance in the cerebellum. Proteins fall into two categories. Up or downregulated in AS compared to controls in cerebellum.

FIGS. 3F, 3G and 3H: Volcano plot of p value vs Log 2 Fold change per brain region. Proteins that are statistically significant in each pairwise comparison are highlighted (Blue: CB, Green: HC, Yellow: Cortex). Proteins significant in all three-brain regions are marked in black stars. Subset of proteins of interest from FIG. 1E is labeled.

FIGS. 31, 3J and 3K: Log 2 Fold change correlation plots between mice cortex and rat cortex for proteins in the amino-acyl t-RNA synthases pathway (FIG. 31), Proteasome subunits (FIG. 3J) and synaptic proteins (FIG. 3K) as filtered in FIG. 2F. Correlation coefficients are calculated using Pearson's method.

FIGS. 4A-4F: Reinstatement of UBE3a in both juvenile and adolescent AS mice rescues protein and pathway alterations

FIG. 4A: Schematic representation of experimental design. Control mice (WT; CreERT2+), AS mice (Ube3aStop/+; CreERT2−), and mice with Ube3a reinstatement (Ube3aStop/+; CreERT2+) were injected with tamoxifen at P21 or P56 and sacrificed at P84. Cortical tissue of both control and AS mice was pooled to generate a sample-specific spectral library in DDA (data dependent acquisition) mode. Individual samples were further analyzed in data-independent acquisition (DIA) mode.

FIG. 4B: UBE3A raw protein intensity plot in cortices of P21 and P56 injected groups of control mice, AS mice, and mice with UBE3A reinstatement, plotted as percentage of P21 control protein levels (mean±S.E.M.).

FIG. 4C: Partial least square-discriminant analysis (PLS-DA) performed on the total proteome of control and AS mouse cortices resolved according to timepoint of UBE3A reinstatement (T1; P21 and P56) and genotype (T2; control, AS, and reinstatement).

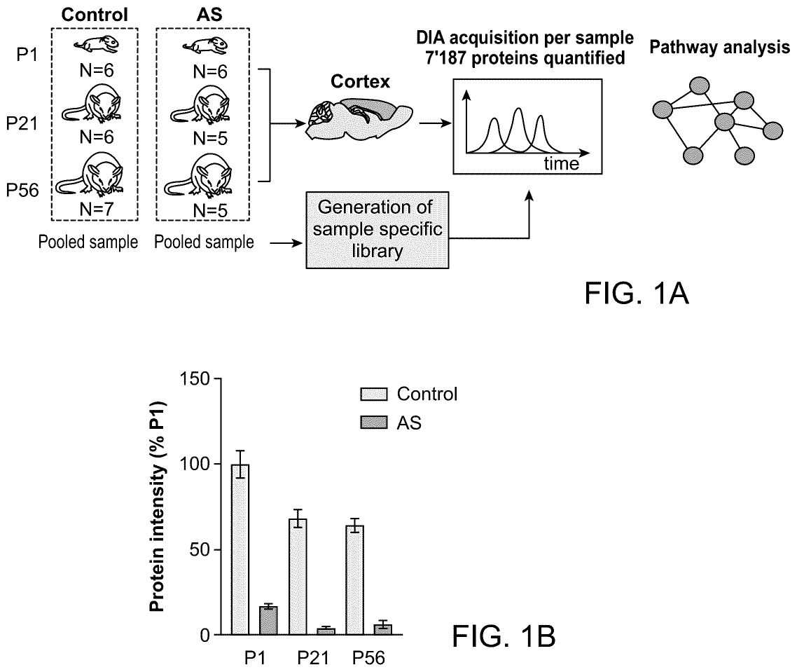

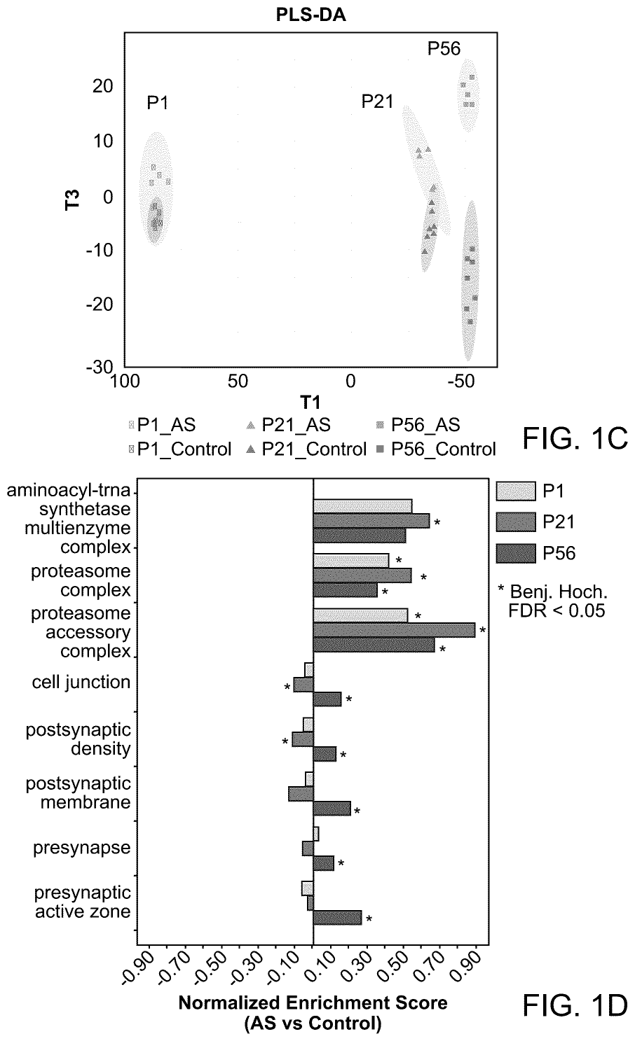

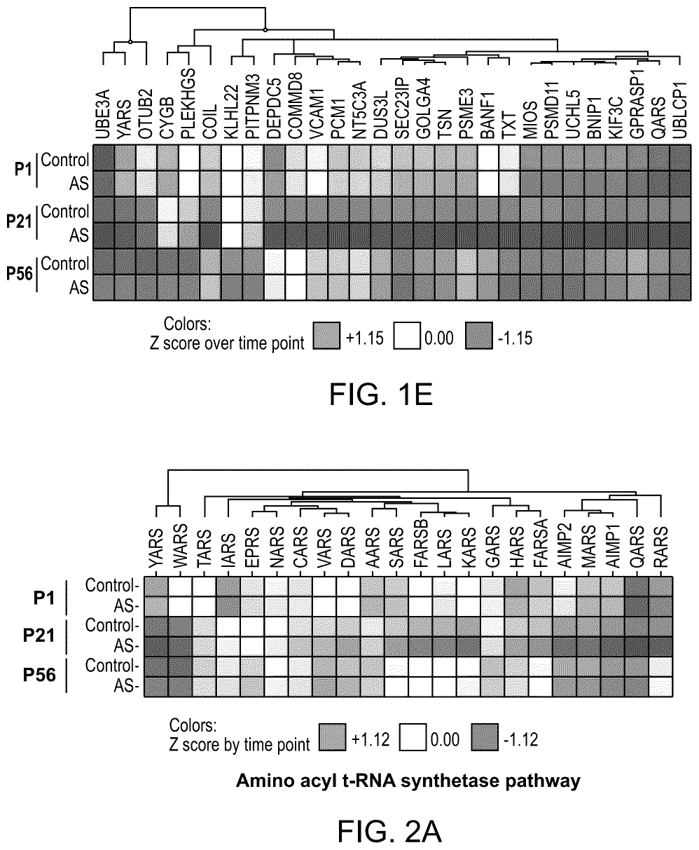

FIG. 4D: Pathway enrichment plot depicting normalized enrichment scores using 1D annotation function using GO: Cellular component genesets in AS vs. control mice (blue), AS mice with Ube3a reinstatement at P21 vs control (red) and AS mice with UBE3A reinstatement at P56 vs control (blue). Select pathways are visualized as observed in FIG. 1D.

FIG. 4E: Heatmap of significantly altered hits between any of the four conditions using ANOVA (adj. p value<0.05). Colors represent average Z-scored protein intensities for each protein.

FIG. 4F: Orthogonal validation of UBE3A targets in an independent sample set of control mice (N=3), AS mice (N=4), and mice with UBE3A reinstatement at P21 (N=4) with capillary western blotting. Statistical analysis was performed using one-way ANOVA followed by Tukey's post-hoc test. (* p<0.05, ** p<0.01, **** p<0.0001).

FIGS. 5A-5H: Transketolase is a direct nuclear target of UBE3A deregulated in rodent and human AS disease models

FIG. 5A: Capillary western blot analysis of Ube3a targets in control, control+UBE3A KD ASO, and AS lines of hiPSC-derived neurons. N=3 for all samples. Statistical testing was performed using one-way ANOVA followed by Tukey's post-hoc test. (* p<0.05, ** p<0.01, *** p<0.001 **** p<0.0001).

FIG. 5B: Immunocytochemical images of transketolase (TKT) and the neuronal marker MAP2 in hiPSC-derived neurons of control, control+UBE3A KD ASO, and AS lines. Nuclei were counterstained with DAPI. Scale bars: 25 μm.

FIG. 5C: Quantification of nuclear TKT signal in neurons (MAP2-positive) and non-neuronal cells (MAP2-negative). Individual data points from 8 images taken from different wells in two independent experiments (neuronal differentiation and ASO treatment) were plotted. Statistical analysis was performed using Kruskal-Wallis test followed by Dunn's post-hoc test.

FIG. 5D: Immunohistochemical images of transketolase (TKT) and the neuronal marker NEUN in the primary visual cortex of adult control and AS rats. Nuclei were counterstained with DAPI. Scale bars: 25 μm.

FIG. 5E: Quantification of nuclear TKT signals in neurons (NEUN-positive) and non-neuronal cells (NEUN-negative). Individual data points from 3 images per animal were plotted (control: N=3, AS: N=2 animals). Statistical analysis was performed using Kruskal-Wallis test followed by Dunn's post-hoc test.

FIG. 5F: Bacterial ubiquitination assay for TKT and RING1B.

FIG. 5G: Capillary western blot analysis of TKT in mice lacking the nuclear (Iso3 KO) or the cytosolic (iso2 KO) UBE3A. (* p<0.05, **** p<0.0001)

### EXAMPLES

AS Mice Exhibit Proteomic Alterations at Birth, which Exacerbate Through Postnatal Development

Data independent acquisition (DIA) mass spectrometry-based proteomics has emerged as the method of choice for label free quantitative proteomics, due to its increased reproducibility, depth of coverage, and high dynamic range compared to classical data dependent acquisition (DDA) [17, 18]. We chose DIA to quantify proteomic changes over the course of mouse cortical development at postnatal day P1, P21, and P56 from control and AS mice. A pool sample of control and AS cortices from all time points was generated, fractionated and measured in DDA mode, leading to a sample specific spectral library containing 8,270 proteins (77,439 unique peptides). Next, individual samples were measured in DIA mode and DIA data was analyzed using the sample specific library to quantify 7,187 proteins across all samples. Protein level data was then subjected to differential protein expression profiling analysis at each time point and pathway enrichment analysis was performed (FIG. 1A). Observed median biological percent coefficient of variations (% Cov) for proteins in each condition was between 11-15% consistent with reports in literature about DIA [17].

UBE3A protein was significantly reduced (<20% of control levels, adj. p value <0.05) in AS compared to control mice at all time points. UBE3A expression levels reduced from P1 to P56 in both controls and AS, consistent with the observation that full silencing of the paternal UBE3A allele in neurons occurs during postnatal development [19, 20] (FIG. 1B). Partial least squares discriminant analysis of all proteins (PLS-DA) separated the samples by both age (T1) and genotype (T3), with P21 and P56 being significantly different from P1. Separation along T3 revealed that AS mice progressively diverge from control mice in terms of their proteomic profiles, with the largest differences in the adult brain (P56) (FIG. 1C).

Pathway enrichment analysis on all proteins using GO: Cellular component (GO:CC) revealed an up-regulation of the aminoacyl-tRNA synthetases and proteasome complex at all postnatal stages, while pre and post-synaptic pathways were significantly altered at P56, coincident with the maturation of synapses (FIG. 1D). Next, we aimed to identify proteins that are differentially regulated at each developmental stage and to observe their differences over time. 28 proteins (FIG. 1E) were significantly (q-value<0.05) regulated between control and AS mice at any time point. Hierarchical clustering of these proteins revealed a genotype and developmental stage dependent effect which proteins both co-regulated and inversely regulated with respect to UBE3A expression. While some proteins like the metabolic enzyme TKT (transketolase) were altered only at P21 and P56, several others like MIOS (a component of the GATOR2 complex), microtubule protein KIF3C, ovarian tumor domain (OTU)-containing deubiquitinating cysteine protease OTUB2 show dysregulation at all time points (FIG. 1E, FIG. S2).

Interestingly, abundance of proteins belonging to the aminoacyl-tRNA synthetase pathway (QARS, DUS3L, YARS) was both increased and decreased in AS at different time points, while proteasomal subunits, UBLCP1, UCHL5, PSME3 were increased at all time points, warranting further investigation of these pathways.

Developmentally Regulated Pathway Alterations in Amino-Acyl tRNA Synthetases, Proteasome, and Synapse in AS Mice

We next examined the trajectory of individual proteins within altered pathways. Aminoacyl-tRNA synthetases (ARS) are evolutionarily conserved enzymes involved in the ligation of amino acids to their cognate tRNAs and occur either free or as part of the ARS

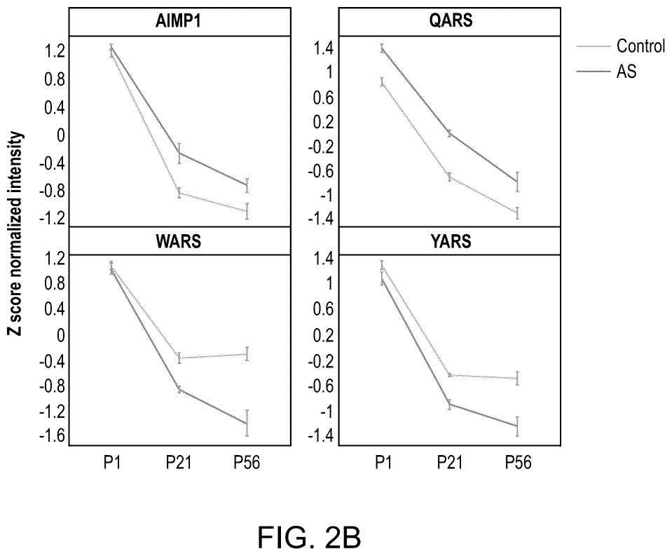

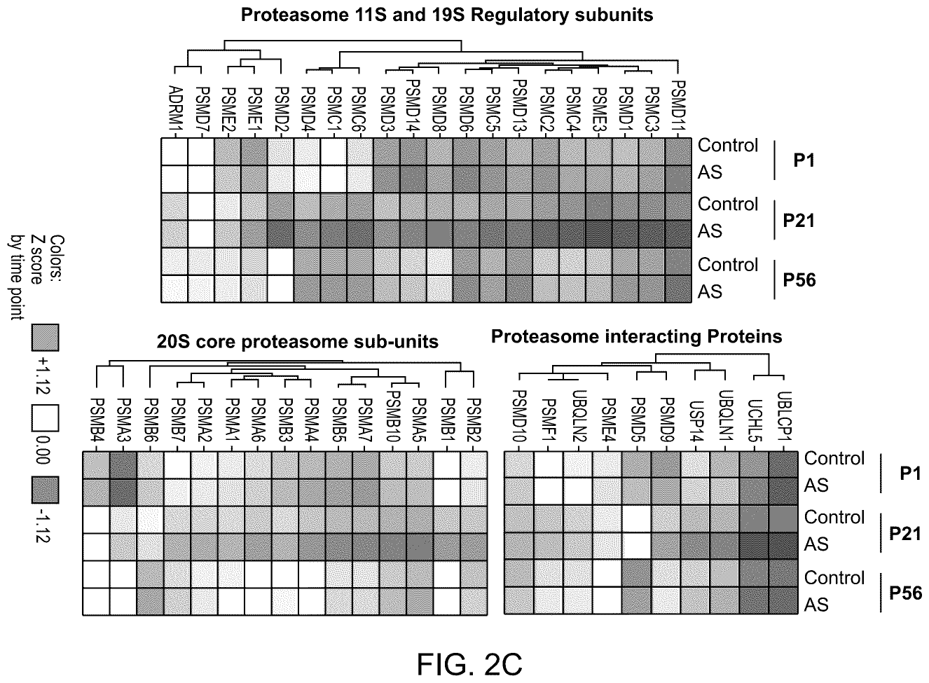

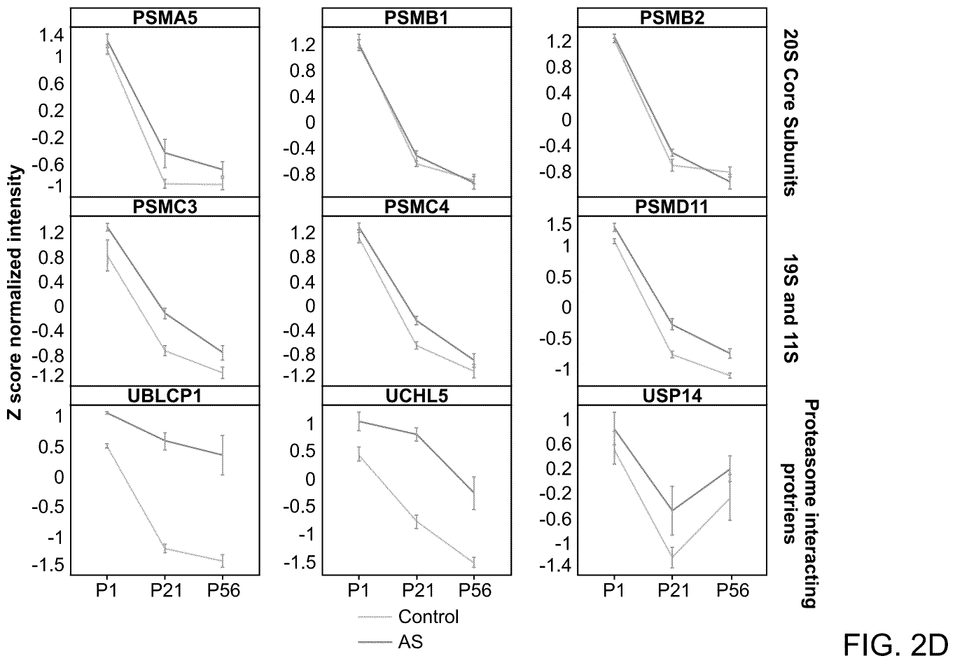

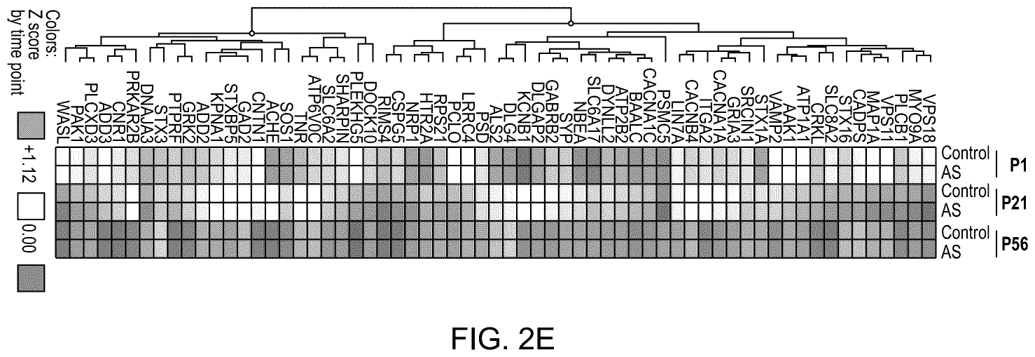

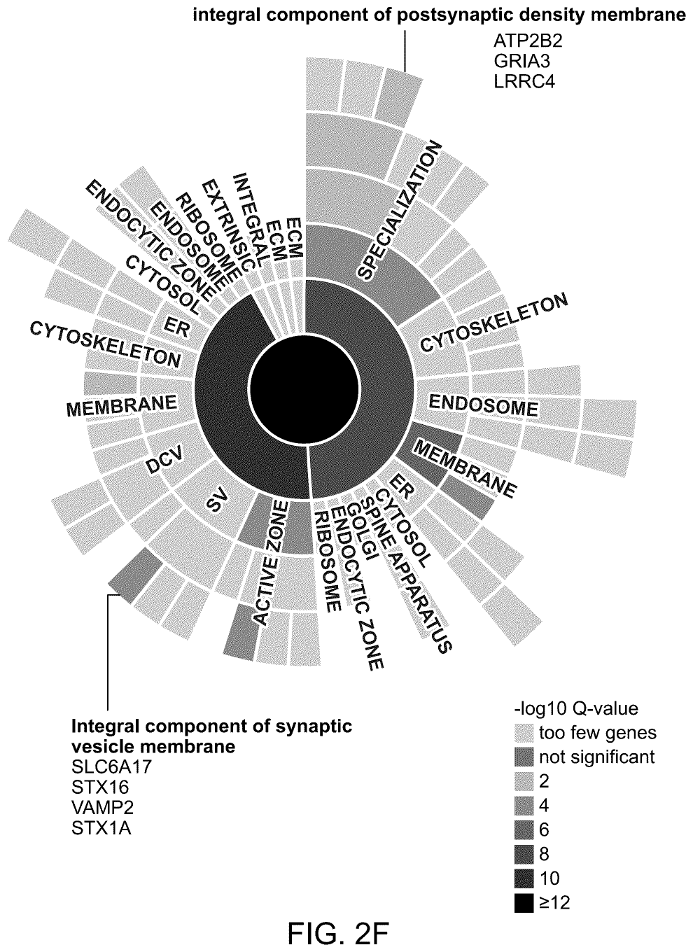

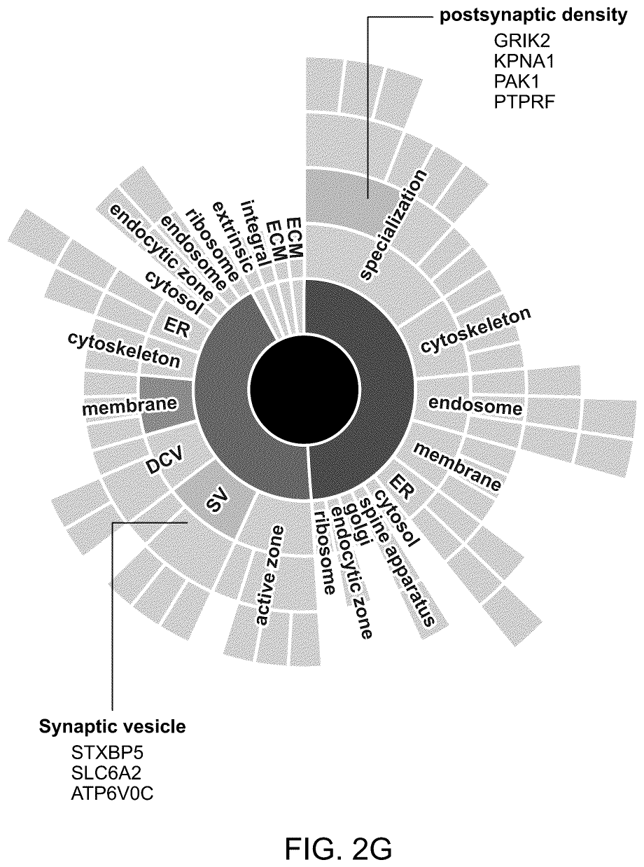

multi-enzyme complex (MSC) [21]. We examined the expression of all ARS and MSC proteins across brain maturation (FIG. 2A), which revealed that Class I ARS proteins, specifically MARS, QARS, RARS, AIMP1 and AIMP2, which belong to the MSC, are increased in AS. Conversely, ARS proteins involved in aromatic amino acids loading, namely tryptophanyl-tRNA synthetase WARS, and tyrosyl-tRNA synthetase YARS, were decreased in AS (FIG. 2A). Interestingly, irrespective of genotype there is a strong reduction of these proteins during postnatal brain development, with disease alteration diverging dramatically at P21 and P56 in AS (FIG. 2B).

Proteins corresponding to all components of the proteasome were altered from birth and persistent to adulthood. Following individual proteins of the proteasome machinery, including the subunits of the 11S and 19S regulatory particle, the 20S core subunits, and

proteasome interacting proteins, revealed disease specific molecular trajectories (FIG. 2C). We observed a consistent increase in abundance of several proteasome interacting proteins (UCHL5, UBLCP1, USP14), as well as of proteins belonging to the 11S and 19S regulatory subunits (PSMD11, PSMC3, PSMD1, PSME3), while the changes in the 20S core proteasome subunits followed a similar trend but were comparably minor (FIG. 2C, FIG. 2D). Thus, UBE3A results in an overall increase of proteasome subunits and accessory proteins throughout development with the most prominent changes seen for the 11S and 19S regulatory subunits and a subset of proteasome-associated proteins.

Next, we examined synaptic proteins that were selectively altered at P56 in AS mice (AS vs. control, q-value<0.05) but not at P1 or P21, and found 63 proteins that fulfilled these criteria (FIG. 2E). Heat map visualization and hierarchical clustering of these proteins revealed a bi-directional AS genotype effect, with sets of proteins being up-(red cluster, 40 proteins) and down-regulated (blue cluster, 23 proteins) in AS vs. controls. To determine, if these two sets of proteins were enriched in particular synaptic sub-compartments, we performed pathway enrichment analysis using Synapse GO, a high quality, and manually annotated synapse GO database [23]. SynGO CC enrichment analysis for the upregulated set of 40 proteins showed that they distributed on both pre- and post-synaptic sites. Proteins belonging to synaptic vesicles and presynaptic active zone (STX1A, SYP, VAMP2), as well as proteins that are integral components of the postsynaptic density (GRIA3, ATP2B2, LRRC2) were significantly enriched (FDR Adj. p-value<0.05) (FIG. 2F, top; data table S3). SynGO analysis revealed that proteins decreased in AS also belonged to both pre- and postsynaptic compartments. Specifically, synaptic vesicle proteins (STXBP5, SLC6A2, ATP6VOC), and postsynaptic density proteins (GRK2, KPNA1, PAK1, PTPRF) were decreased in AS at P56.

Thus, protein alterations in AS mice are dynamic; ARS and proteasome subunits are altered from birth, while many changes in synaptic proteins develop over final stages of brain maturation.

### Adult AS Rats Recapitulate the Proteomic Alterations Observed in AS Mice Across Different Brain Regions.

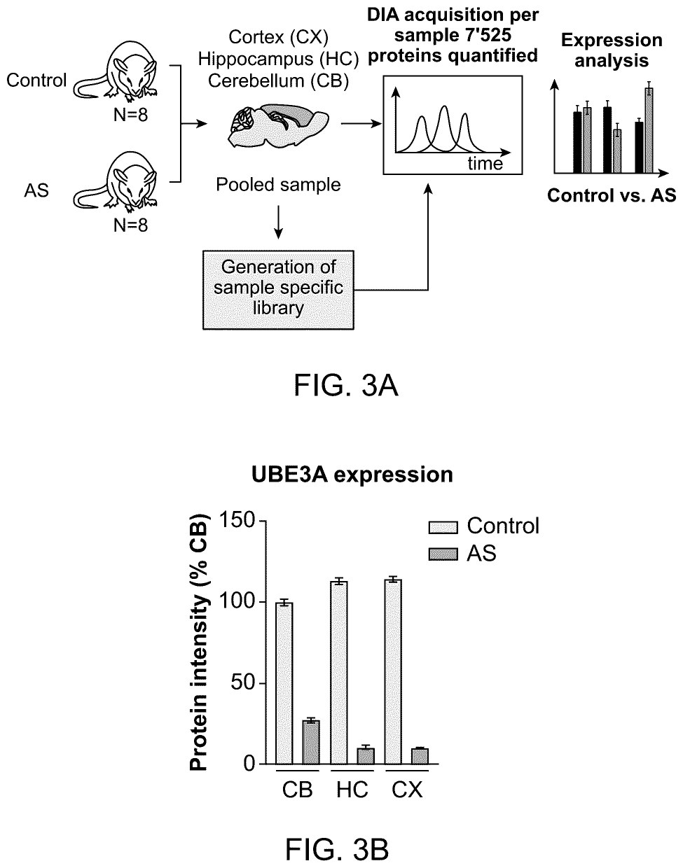

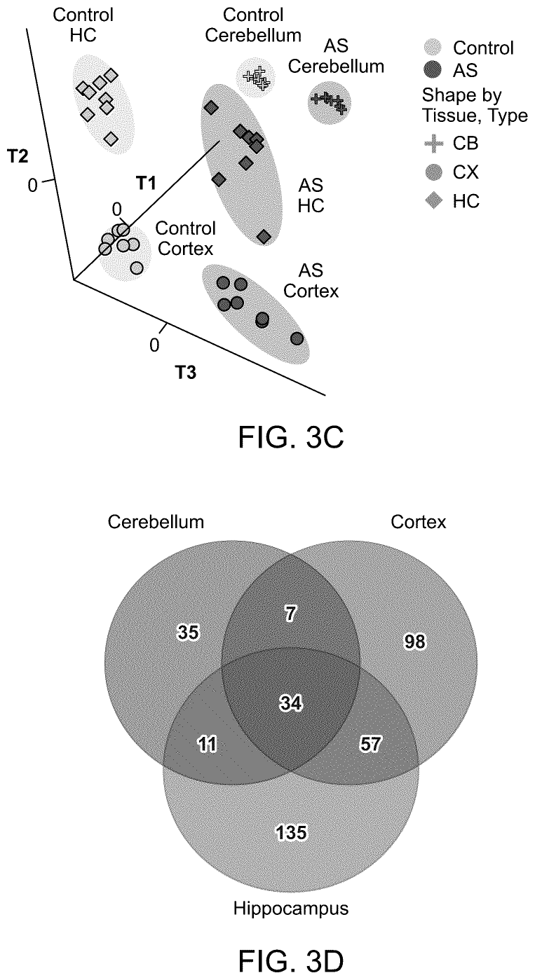

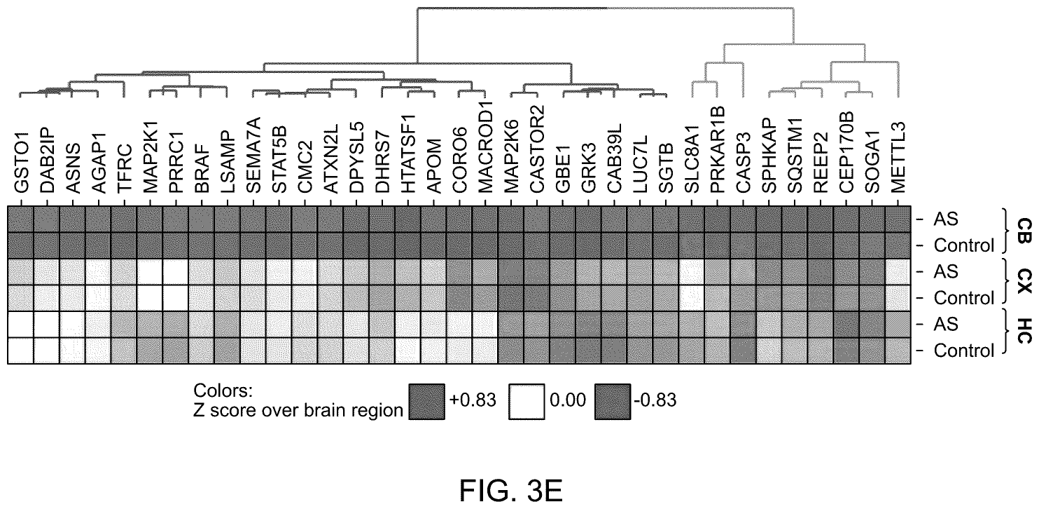

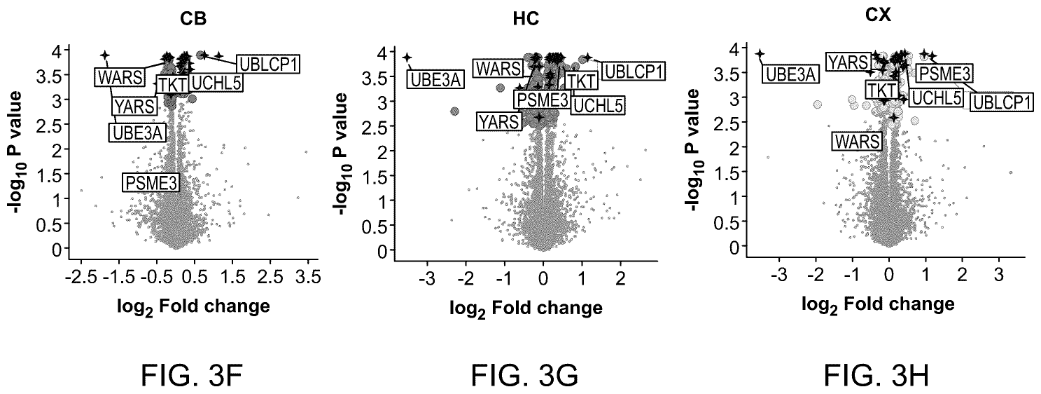

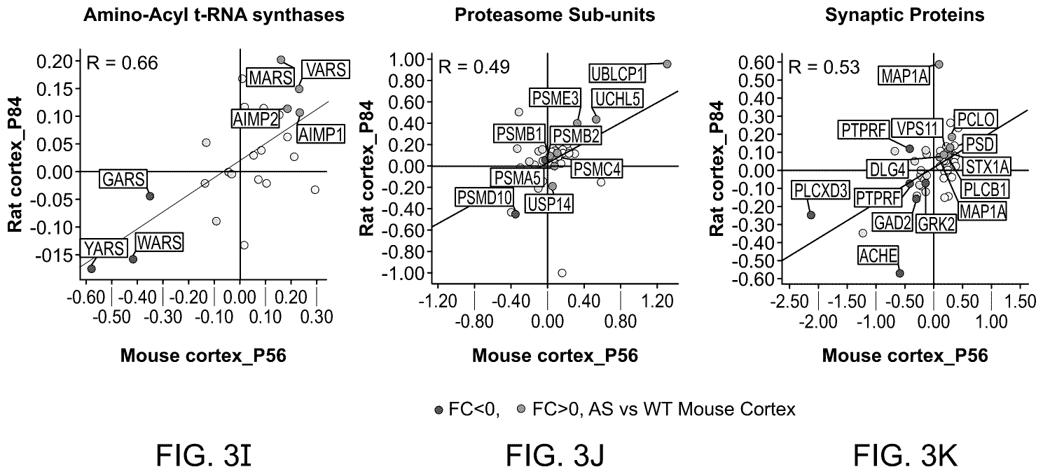

We next explored if the observed alterations are conserved across species and brain regions in an AS rat model, by making use of a newly available AS rat model [24]. We performed DIA based LC-MS analysis of three rat brain regions (CB: Cerebellum, HC: Hippocampus, and CX: Cortex) in adult (P84) control and AS rats (FIG. 3A). A hybrid spectral library was generated from DDA runs on a fractionated pooled sample of all three brain regions from all samples combined with DirectDIA measurements to generate a library of 8,928 proteins (116,603 unique peptides), allowing us to quantify 7,525 proteins across all samples. Coefficients of variation were around 10% per sample type. UBE3A expression was robustly reduced in all three brain regions in AS rats (FIG. 3B). Similar to mice [25, 26]. UBE3A levels in the cerebellum of control rats were lower compared to hippocampus and cortex, while residual UBE3A levels in the AS rat cerebellum were higher (27% of control levels in cerebellum vs. <10% of control levels in hippocampus and cortex; (FIG. 3B). Subsequent PLS-DA analysis revealed a robust separation between genotypes (T3) and brain regions (T2, T1) (FIG. 3C). The proteomic profile of the cerebellum was distinct to the cortex and hippocampus, which shared more similarities with each other, likely reflecting different developmental origin and cytoarchitecture.

Examination of the proteins significantly altered in the AS rat brains revealed that 34 proteins were changed across all three brain regions, and that there was large overlap in the alterations in the hippocampus and cortex (FIG. 3D). Significant hits in the cerebellum were visualized by hierarchical clustering, revealing sets of upregulated (blue cluster) and downregulated (green cluster) proteins (FIG. 3E). Several of the proteins altered in the cerebellum showed directional trends in cortex and hippocampus, but did not meet the statistical cutoffs. Interestingly, several proteins (including GSTO1, DAB2IP, ASNS, AGAP1), were specifically altered in the cerebellum in AS suggestive of cerebellar neuron specific regulation of these substrates. Proteins belonging to proteasomal subunits (including UBLCP1, UCHL5, PSME3) and the ARS proteins YARS and WARS were altered in all three brain regions (FIGS. 3F, G, H, black stars).

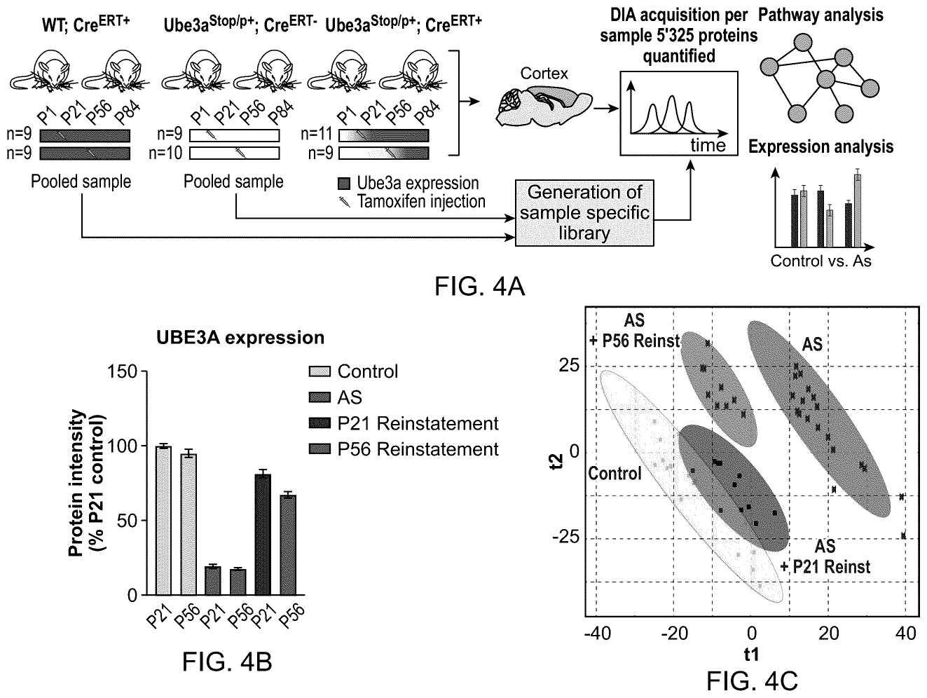

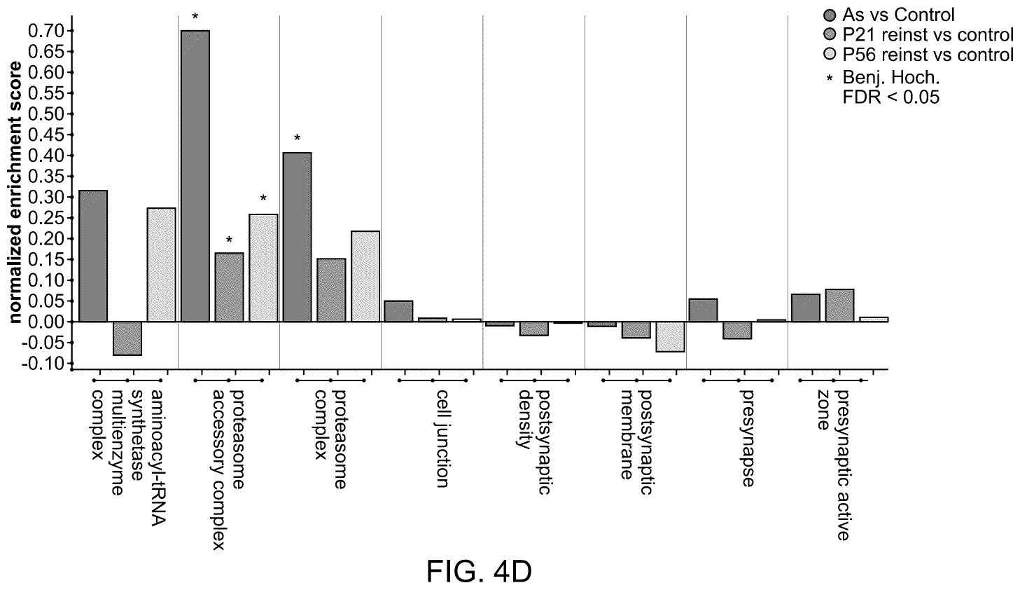

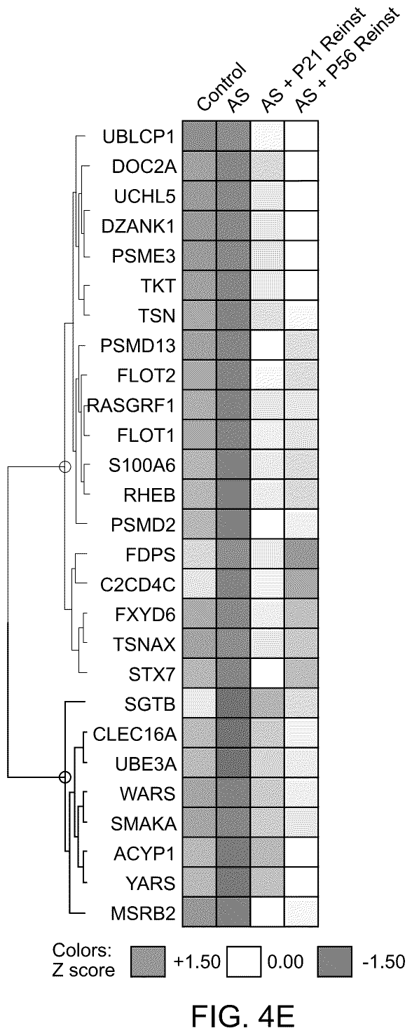

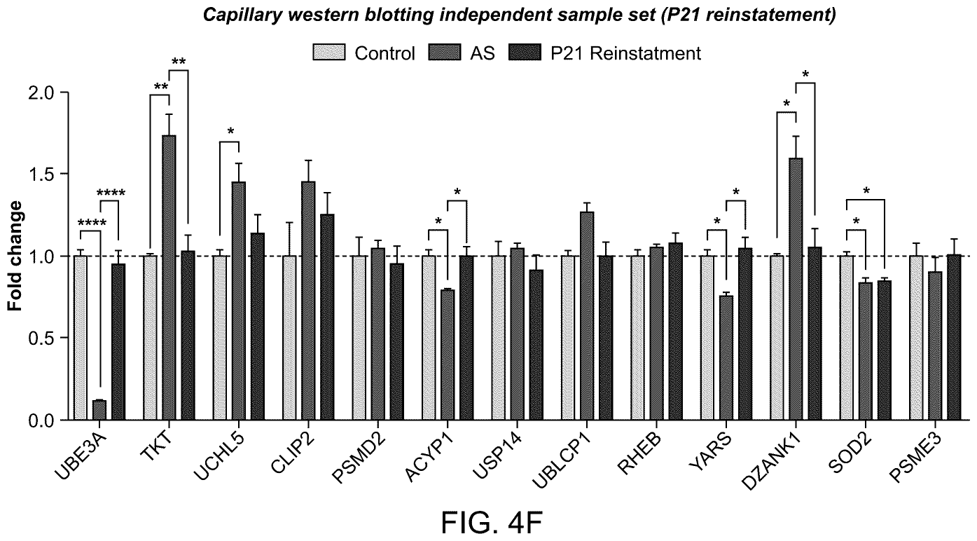

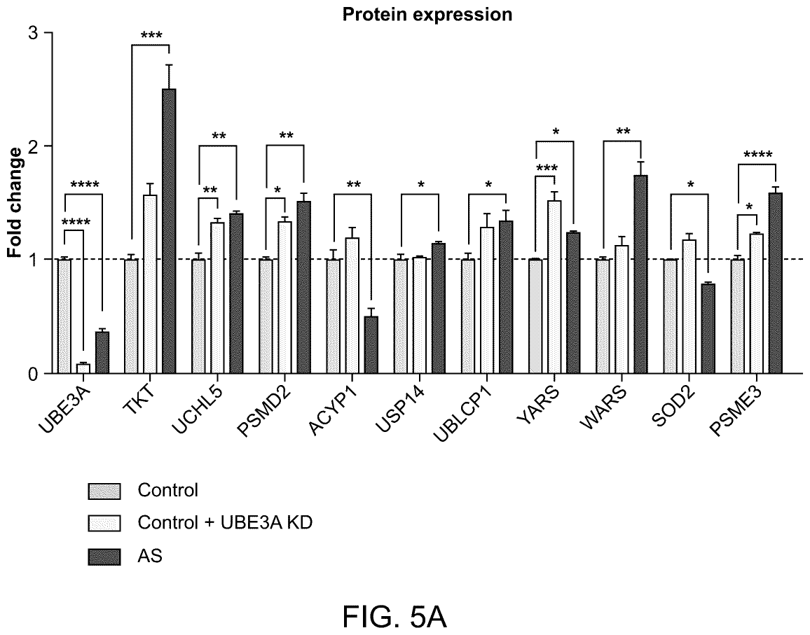

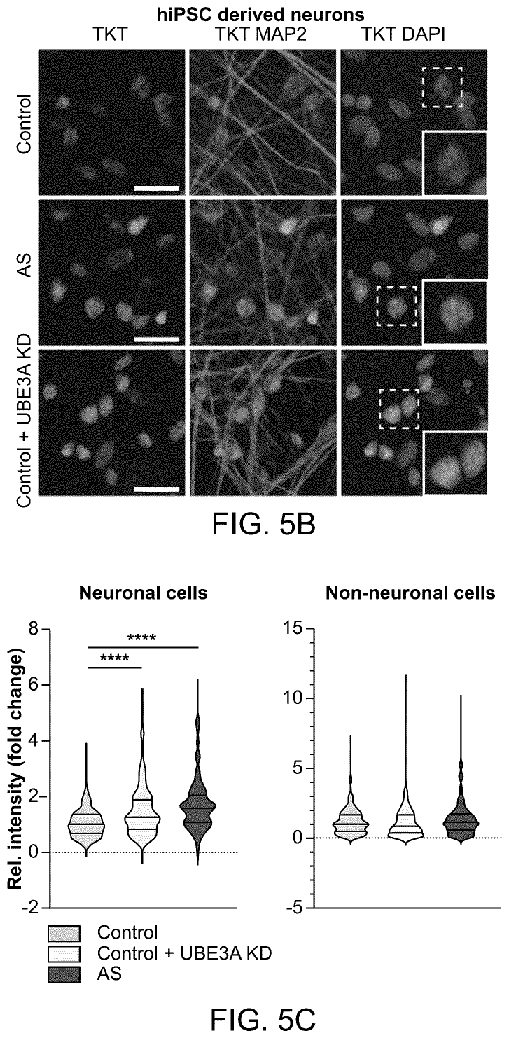

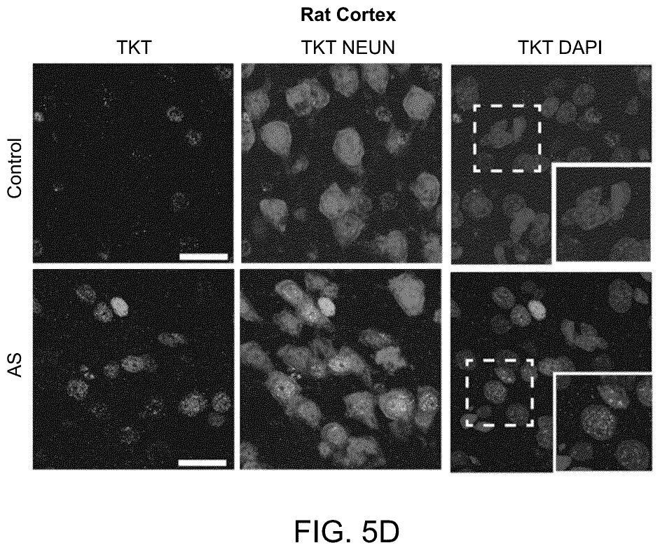

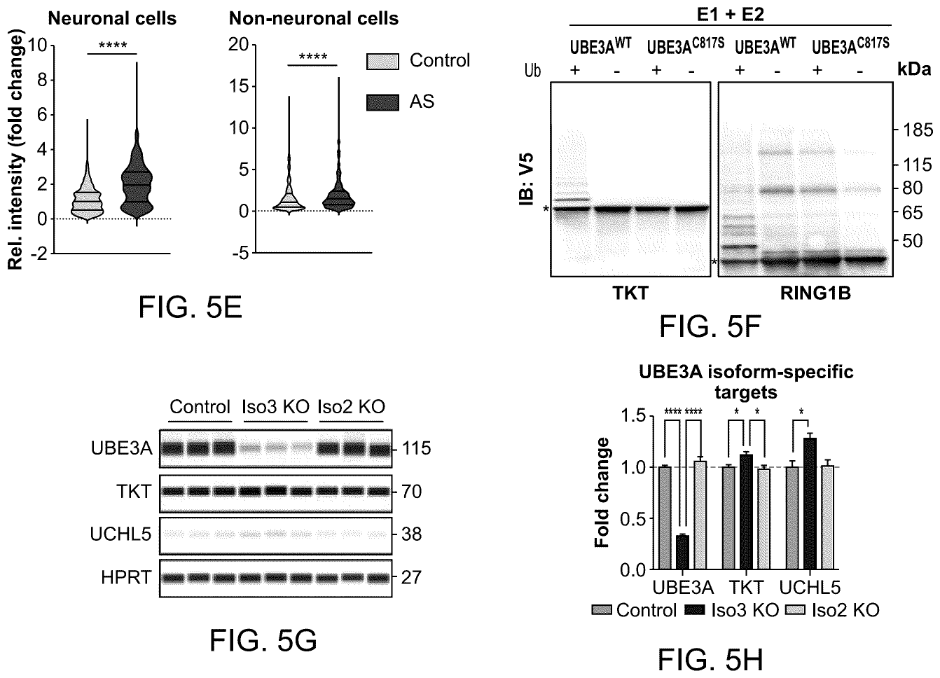

Next, we examined the correlation between altered protein corresponding to the 3 major pathways in AS P56 mouse and rat cortices (FIGS. 31-3K), and across rat brain regions. We subdivided each pathway into sets of proteins that are upregulated (in red) or downregulated (in blue) in AS mice cortex at P56 compared to controls. Correlation plots of fold changes for the ARS pathway revealed a significant correlation between rat and mice (R=0.66, p=4.29E-004), with YARS, WARS and GARS down regulated, and MARS, VARS, AIMP1 and AIMP2 upregulated in AS in both mice and rats (FIG. 31).

Proteasome complex proteins showed a robust correlation between rat and mouse cortices (FIG. 3J) (R=0.49, P=2.12E-004) and across brain regions. Ubiquitin-like domain-containing CTD phosphatase 1; UBLCP1, Ubiquitin carboxyl-terminal hydrolase isozyme L5; UCHL5 and proteasome activator complex subunit 3; PSME3 were consistently upregulated in adult rats across all three brain regions (FIG. 3J). Proteasome assembly chaperone 3; PSME3 and 26S proteasome non-ATPase regulatory subunit 10; PSMD10 were consistently downregulated in AS compared to controls in AS mice as well as in AS rat brain regions. Like for the aminoacyl tRNA synthetase pathway, cortex and hippocampus showed more similarity to each other and the cerebellum diverged slightly.

Analysis of synaptic protein alterations showed both similarities and differences between species and across rat brain regions (FIG. 3K). AS rat cortex was similar to both mouse cortex (R=0.53, P=4.86E-006) and rat hippocampus (R=0.56, P=1.59E-006) while diverging in AS cerebellum (R=0.21, P=8.60E-002). Of interest, the synaptic proteins GAD2, ACHE, PLCXD3 and GRK2 were downregulated in AS in all brain regions and across species, while PSD, DLG4, PLCB1, and VPS11 were upregulated in AS. Others such as MAP1A, PCLO, PTPRF, STX1A showed brain region- or species-specific differences.

Reinstatement of UBE3A expression in juvenile and adult AS mice rescues altered proteomic state to varying degrees

We next explored if these alterations in potential UBE3A targets and pathways could be rescued by UBE3A reinstatement during clinically relevant therapeutic time points, i.e. in juvenile and adult mice. To address whether the altered molecular proteome can be rescued by reinstating UBE3A expression, we utilized an AS mouse model harboring a tamoxifen-inducible UBE3A allele [4]. Control (WT; CreERT2+), AS (Ube3aStop/p+). UBE3A reinstatement mice (Ube3aStop/p+; CreERT2+) were injected with tamoxifen at either P21 or P56, corresponding to juvenile and adult developmental stages, and sacrificed at P84 to compare rescue at the two time points (FIG. 4A). Cortical tissue was used to quantify 5325 proteins across all samples in DIA mode, analyzed with pooled libraries created from both control and AS groups.

UBE3A reinstatement at P21 and P56 was able to rescue cortical UBE3A protein levels from <20% to 81% and 71% of that of control animals, respectively, as measured by LC/MS (FIG. 4B). PLS-DA separated the samples by both time point of reinstatement (T1) and genotype (T2), with control being significantly different from AS mice (FIG. 4C). Global proteomic profiles analysis revealed that the P21 reinstatement group reverted to controls, and

P56 reinstatement partially rescued the AS proteome. These results suggest that AS-related changes in protein homeostasis can be almost completely rescued by UBE3A reinstatement in juvenile brains and to a lesser extent in adults.

We next performed expression and pathway analysis of differentially changed proteins to map trajectories of UBE3A downstream targets between different groups Pathway enrichment analysis on all proteins using GO: Cellular component (GO:CC) analysis comparing AS with control, or AS with reinstatement at P21 or P56, revealed a rescue of aminoacyl-tRNA synthetases at P21 but not P56, and rescue of both the proteasome complex and synaptic protein gene sets at both time points (FIG. 4D).

A heatmap of the top 27 individual proteins that were significantly altered between any of the four sample sets reveals that the majority of proteins could be reverted to some extent in both early and late rescue. (adj. p-value<0.05; (FIG. 4E). Hierarchical clustering was performed according to the degree of co-regulation (magenta) or inverse regulation (green) with respect to UBE3A expression. While the majority of proteins, including top hits belonging to ARS (such as YARS and WARS) and proteasomal subunits or proteasome accessory proteins (e.g. UBLCP1, UCHL5, PSME3) show an enhanced rescue at P21 vs P56; while the expression several synaptic proteins including FDPS, C2CD4C, FXYD6, TSNAX, and STX7 were normalized at P56, indicating that their response to UBE3A can vary.

### Transketolase is a Nuclear Target of UBE3A in Rodents and Humans

To confirm a subset of the top identified UBE3 A targets with an orthogonal method, we performed biochemical analysis of an independent cohort of control, AS, and P21 reinstatement mice cortical samples using capillary western blotting (FIG. 4F). Significant changes in protein levels in control compared to AS mice, and in P21 reinstatement compared to AS mice, were detected for several of the tested hits, including TKT, UCHL5, ACYP1, YARS, DZANK, and SOD2. These results confirm robustness of the identified targets across independent experiments and protein quantification methods. In addition, transcript levels of the top identified hits in cortical tissue of control and AS mice did not differ significantly, indicating that changes in expression levels are at translational level or post-translationally.

We next investigated whether the top UBE3A-dependent hits in rodents are altered in neurons derived from induced pluripotent stem cells (iPSCs) which were generated from AS patients and controls blood (Pandya et al., 2021). After 6 weeks of differentiation, expression of this selected set of proteins was analyzed with capillary western blotting. All proteins tested showed significant upregulation in AS neurons compared to controls, including all of those associated with the proteasome (UCHL5, PSMD2, USP14, UBLCP1, PSME3), and the ARS pathway (YARS, WARS). Furthermore, treatment of control neurons with an UBE3A-targeting ASO to lower UBE3A expression (UBE3A KD) mimicked changes seen in AS patient neurons, albeit with smaller fold changes in most cases (FIG. 5A). Consistently, across AS patient neurons, and AS mouse and rat brains, TKT revealed the largest fold change (FIG. 5A). We thus sought to address the mechanism of UBE3A dependent regulation of TKT.

Distinct from other enzymes in the pentose phosphate pathway, TKT has been previously reported to have a substantial nuclear localization in certain cancer cells and normal tissues, and to contain a nuclear localization signal [28, 29]. Similarly, TKT appeared mostly nuclear in human and rat neurons (FIG. 5B, FIG. 5D). In human neurons, co-labelling with DAPI and MAP2 revealed that TKT expression was higher in the nuclei of neurons compared to non-neuronal cells (MAP2+vs. MAP2-cells) and increased in AS neurons and UBE3A dependent manner FIG. 5B, FIG. 5C. TKT nuclear intensity in both neuronal iPSC-derived AS and UBE3A KD cultures revealed significant upregulation of TKT expression (MAP2-positive nuclei compared to controls, +59%; FIG. 5B, FIG. 5C). In rat cortices, there was a low expression in both neurons (NeuN-positive) and non-neuronal cells. Consistent with the patient neurons, nuclear TKT signal was significantly upregulated in the AS condition (+95%; FIG. 5D, FIG. 5E). In rat brains, there was a small increase in non-neuronal cells, which could be due to UBE3A gene dosage reduction (FIG. 5E).

To assess if TKT is a direct target of UBE3A, we used a previously described cellular ubiquitination assay performed in E. coli [14, 30]. To this end, E. coli cells were transformed with plasmids encoding the rabbit ubiquitin-like modifier activating enzyme 1 (UBA1), E2 ubiquitin-conjugating enzyme UBCH5, E3 ligase UBE3A (or the catalytically inactive variant), ubiquitin, and TKT or RING1B. RING1B is a well-established target of UBE3A and serves as positive control in this assay [14, 31]. The presence of active UBE3A and all components of the ubiquitination cascade leads to formation of slower migrating bands, for both RING1B and TKT (FIG. 5F). These slower migrating bands are not seen when ubiquitin is absent or in the presence of the catalytically inactive UBE3AC817S. These results strongly suggest that UBE3A can directly ubiquitinate TKT.

UBE3A has been shown to be expressed in several isoforms that differ from each other in cellular localization. In humans, three functional isoforms exist that vary at the N-terminus [32, 33], while in mice there are only two, a shorter, predominantly nuclear isoform (mUBE3A-Iso3), and a longer, cytosolic isoform (mUBE3a-Iso2). Furthermore, loss of the nuclear UBE3 A isoform has been shown to be sufficient to induce AS phenotypes in mice [13]. Given the UBE3A-dependent upregulation of TKT in neuronal nuclei, we asked whether its regulation is UBE3A isoform-specific. Lysates from cortical tissue of mUBE3A-Iso3 and mUBE3a-Iso2 knockout mice and their respective controls were analyzed with capillary western blotting. Since mUBE3A-Iso3 accounts for the majority of UBE3A protein, knockout of this isoform reduced the total UBE3A levels by over 60% (FIG. 5G). Compared to wild-type controls, levels of both TKT and UCHL5 (a known UBE3A target localized to the nucleus) were upregulated in tissue of mUBE3A-Iso3 knockout mice (+12.8% and +28.4%, respectively), while knockout of the cytoplasmic isoform mUBE3a-Iso2 did not significantly affect expression levels (FIG. 5G). This suggests that TKT protein levels are controlled by the nuclear isoform mUBE3A-Iso3.

### Discussion

In this study, we comprehensively mapped UBE3 A-driven temporal and spatial changes of the rodent proteome. Together with our previous proteomic analysis of AS patient derived neurons, we highlight disease alterations across species. We demonstrate that the AS proteome exacerbates over the course of postnatal development and that it can be reverted by UBE3A reinstatement at juvenile and young adult postnatal stages, albeit rescue being more effective at the earlier time point. In addition, we identified three major cellular pathways affected by UBE3A loss in mice that were also observed in rat and human models of AS: the proteasome, ARS, and several synaptic pathways. These results imply that the regulation of fundamental global cellular processes is controlled by UBE3A and that UBE3A alteration exhibits different developmental trajectories.

The most striking change observed in the AS proteome in all species and across all developmental stages, was the upregulation of proteasomal subunits and proteasome accessory proteins. It has been reported previously that UBE3A can directly interact with the 26S proteasome and regulate turnover of proteasomal subunits [13, 16, 34, 35]. Our data shows that changes in protein abundance affected the 19S and 11S regulatory subunit and proteasomal accessory proteins to a larger extent than the 20S core. In a non-neuronal context, several top hits, including polyubiquitination receptor PSMD4, deubiquitinase UCHL5, and regulatory subunit PSMC3 could be directly ubiquitnated by UBE3A, and in turn could negatively affect proteasome function including binding and process its substrates [36]. It is thus conceivable that UBE3A loss affects proteasomal function in a similar way in vivo, which will lead to cumulative effects on the AS proteome. In addition, changes in accessory protein abundance and function may directly contribute to the disease phenotype by hampering the recognition of specific disease relevant substrates in neurons. Future studies should address to what extent Ube3a proteasomal dysfunction as well as nuclear vs cytoplasmic Ube3a contribution of to the widespread proteome disease changes.

Similar to the proteasome pathway, we observed changes in ARS across all developmental time points. ARS are a family of nuclear-encoded enzymes that ensure correct translation by conjugating amino acids to their cognate tRNA molecule providing a key initial step for protein translation [21]. Similar to the proteasome, ARS plays a central role in protein homeostasis, in this case translation but, in contrast to proteasome subunits, the association with UBE3A-dependent mechanisms is less clear. Comprehensive network analysis using human protein-protein interaction databases identified several ARS as part of the UBE3A interactome, including AIMP1, AIMP2, MARS and QARS, although none of them have been reported to act as direct substrates [37]. This is consistent with our findings that individual proteins in the ARS pathway are not universally upregulated. While protein abundance QARS and AIMP1 proteins were elevated, YARS and WARS were decreased in AS mice which suggests a more complex UBE3A-dependent regulatory mechanism with both direct and indirect effects. Over the course of eukaryotic evolution, multicellular organisms have bear additional ARS protein domains that fulfil functions beyond translation [38]. Whole-exome and whole genome sequencing in patients with unknown etiologies connected loss-of-function mutations in ARS genes, such class-I ARS proteins YARS and WARS, to nervous system dysfunction, developmental delay, and epilepsy [39, 40]. Mutations in the catalytic domain, reducing the aminoacylation rate or accuracy of tRNA recognition by the ARS, may interfere with protein synthesis and could thereby contribute to many of the associated disorders. Furthermore, tyrosine and tryptophan are crucial building blocks of serotonin and dopamine, which are essential for normal synaptic function, and contribution of neurotransmitter imbalance to AS behavioral phenotypes have been reported in AS patients and mouse models of AS [41]. The existence of disease-causing ARS mutations that do not interfere with catalytic functions indicates that non-canonical ARS functions can also contribute to pathological phenotypes. It is conceivable that changes in abundance of individual ARS proteins in AS affect MSC composition and translation-independent roles such as mitochondrial homeostasis, nuclear rRNA synthesis, and cytokine stimulation [42]. Future work will be needed to determine whether translational mechanisms or other ARS associated functions are disturbed in AS and how this contributes to the disease phenotype.

In contrast to proteasome and tRNA synthase pathways, synaptic hits were mostly altered at later developmental stages coincident with the full maturation of the nervous system. Many UBE3A downstream targets involved in neuronal function have been characterized before, although for most of these targets it remains to be determined if they are direct substrates. In our dataset, abundance of a wide range of synaptic proteins were either increased or decreased confirming dysregulation of the synapse at the molecular level. Our analysis revealed that hits localized to both pre- and postsynaptic compartments, including integral components of synaptic vesicles, presynaptic active zone, and postsynaptic density, indicating overall synaptic perturbations rather than changes in a single synaptic compartment. It remains to be determined how changes in abundance of each individual component affect neurological function and what is the extent of crosstalk directly via UBE3a or as consequence pathways such as the proteasome or ARS which manifest at an earlier developmental stage.

Recently, a novel rat model of AS was generated that harbors a deletion of the maternal UBE3A gene and recapitulates many of the behavioral phenotypes previously observed in AS mice [24]. Our study represents the first proteomic study of AS rat brain tissue, and we confirmed top hits identified in AS mice, including members of the ARS family YARS and WARS, proteasomal subunits and accessory proteins such as UBLCP1 and UCHL5, and the metabolic enzyme TKT, which showed consistent upregulation across all our AS datasets. Alterations in proteasome, ARS, and synaptic pathways in rat AS cortices robustly correlated with P56 AS mice, indicating that UBE3A-dependent pathway alterations are conserved, pointing towards broad underlying disease mechanisms in AS.

Individual brain region analysis in rats identified commonality and regional differences between cortex, hippocampus, and cerebellum; areas that show functional impairment in AS patients and have been the primary focus of research over the years [43]. Investigating molecular mechanisms underlying potential region-specific dysfunction such as cognitive and learning disabilities, ataxia, and motor incoordination, is of particular interest. Interestingly, conserved proteins and pathways between AS mice and rats were also confirmed to be altered across different brain regions. Interestingly, the cerebellar AS proteome diverged from the cortex and hippocampus and this might have implications in motor dysfunction and ataxia seen in rodent models and AS patients. Cerebellar neuronal cytoarchitecture is quite unique, the majority of neurons, cerebellar cells, hardly express UBE3A, and thus the possible UBE3A protein changes could originate from Purkinje or Golgi cells [26]. Several of the AS cerebellar specific proteins are either exclusively expressed, or show significant enrichment in the cerebellum compared to other brain regions. DPYSL5 is an enzyme expressed in the developing brain where it regulates neurite outgrowth via interaction with actin [44]. In the adult cerebellum, it regulates dendritic development and synaptic plasticity of Purkinje cells in mice [45]. Anti-CRMP5 antibody is often detected in serum of patients with subacute cerebellar ataxia and proteomic profiling revealed significantly increased expression in patients with cortical dysplasia and epilepsy [47]. BRAF kinase gain-of-function mutations are associated with cardiofaciocutaneous syndrome (CFC) which presents a range of neurological phenotypes that resemble AS, including developmental delay, intellectual disability, and seizures. In iPSC-derived neurons, BRAF gain-of-function leads to premature neuronal differentiation and depletion of the progenitor pool, and neurons generated in these cultures showed higher intrinsic excitability [48]. Lastly, loss of function of the asparagine synthetase ASNS is associated with microcephaly, most likely caused by decreased proliferation of progenitors [49]. ASNS metabolically connects the four amino acids L-aspartate, L-asparagine, L-glutamate, and L-glutamine and therefore a dysregulation of the balance of these amino acids in the brain might contribute to microcephaly and brain malfunction.

Although larger deletions of the UBE3A locus are observed in AS patients, loss of UBE3A expression alone has been shown to be sufficient to induce AS. Therefore, restoring UBE3A expression is a promising therapeutic strategy currently undergoing clinical trials. In a conditional AS mouse model harboring an inducible maternal UBE3A allele, age-dependent rescue of specific behavioral phenotypes was demonstrated after UBE3A reinstatement [4, 50]. Moreover, although a single intracerebroventricular injection of an UBE3A ATS ASO in adult AS mice failed to rescue most behavioral phenotypes [7]. ASO injection of newborn mice rescued many behavioral phenotypes (Milazzo in press). While rodent behavior suggests that UBE3A reinstatement is more effective at early time points, this may not necessarily be the case for molecular/proteome changes. Ube3a neuronal loss at early stages of development can cause developmental alterations which are difficult to dissect from direct or indirect protein modulation in mature neurons. We therefore asked to what extent the AS proteome in mice could be reverted by reinstating UBE3A in adolescence (P21) and adulthood (P56). Strikingly, restoring UBE3A expression at either stage restored proteomic homeostasis to a significant degree, albeit P21 rescue being more efficient. To a large extent, proteasome and synaptic pathway alterations were reverted at both time points, and this is in line with the observation that most electrophysiological parameters can also be restored upon adult UBE3A reinstatement [51]. These results highlight that disease trajectories of proteins and associated pathways are UBE3A dependent can be restored after birth. Interestingly, some proteins were only modulated in the adult rescue, suggestive of a role in fully mature neurons. P56 specific synaptic hits included TSNAX, a signaling protein critical for synaptic plasticity [52], and STX7, a component of synaptic vesicles (SV) that may play a role in maintaining a presynaptic recycling pool of SV [53]. These results demonstrate that restoration of UBE3A expression in early adulthood can significantly revert the AS proteome and potentially rescue proteasome and synaptic function, providing further encouragement for the advancement of disease-modifying therapies in AS.

In a previous study, proteomic analysis of patient derived iPSC neuronal cultures and after UBE3A reinstatement using ASOs revealed a set of human specific UBE3 A targets, including the GAG domain containing protein PEG10 [27]. In addition, several top hits identified in the patient neurons were also altered in AS models, including PPID, DST, and UCHL5. Several additional proteasome and ARS proteins were in AS neurons and phenocopied by knockdown of UBE3A. Notably, TKT, the protein showing the largest change across rodent models and validated as a direct UBE3A target, was also the most altered in human AS neurons. TKT is the rate limiting enzyme of the pentose phosphate pathway (PPP), a metabolic pathway that generates NADPH and the building blocks for nucleotide synthesis. Furthermore, TKT is part of the non-oxidative branch of the PPP that connects it with glycolysis and due to its reversible nature, it can control the flux through the PPP. TKT function is dependent on the coenzyme thiamine, and thiamine deficiency as well as rare mutations in the TKT gene underlie a range of neurological dysfunctions, although the precise mechanism by which TKT loss of function leads to these phenotypes is not well understood [54, 55]. Intriguingly, TKT localization in both human neurons and rat brain tissue was found to be predominantly nuclear, even though the PPP takes place in the cytosol [56]. Using a mouse model harboring a deletion of either the nuclear or cytoplasmic isoform of UBE3A (ref), we demonstrated that TKT is directly regulated by nuclear UBE3A. The tight regulation of TKT by nuclear UBE3A and upregulation in AS condition raises the question whether 1) regulation by UBE3A is directly linked to its metabolic function and the PPP, or 2) unknown non-canonical functions of TKT in the nucleus exist, and 3) if overabundance of TKT contributes to the disease phenotype. Non-canonical regulatory functions have been described for many metabolic enzymes, including glycolytic enzymes that can act as protein kinases and transcriptional regulators [57], and in line there is a report that nuclear TKT interacts with EGFR functionally independent of TKT enzymatic activity [28]. Future studies are warranted to elucidate whether TKT has a role AS pathophysiology.

Together with our previous proteomic analysis of AS-patient derived neurons [27], our work provides a proteomic resource to aid in biomarker and translational research. Future efforts will be required to determine the altered human proteome from AS brain postmortem samples and analysis of patient CSF samples to determine if TKT and other proteins are secreted from neurons in disease, and to validate it as a downstream UBE3A biomarker.

In summary, we provide proteomic datasets from 143 runs across 3 different AS rodent models: 1) in mouse cortices across different developmental stages, 2) rat cortices and other disease relevant brain regions, and 3) in mice after UBE3A rescue; and provide a comprehensive proteomic atlas of UBE3A-dependent protein and pathway alterations.

### Materials and Methods

### Animals

Mice were housed in individually ventilated cages (IVC; 1145 T cages from Techniplast) in a barrier facility. All animals were kept at 22±2° C. with a 12 h dark and light cycle, and provided with mouse chow (801727CRM(P) from Special Dietary Service) and water ad libitum. All animal experiments were conducted in accordance with the European Commission Council Directive 2010/63/EU (CCD approval AVD101002016791).

### Data-Independent Acquisition (DIA) Mass Spectrometry

Total protein profiling of rat and mouse tissue was performed at Biognosys AG (Schlieren, Switzerland) using Biognosys' Hyper Reaction Monitoring (HRM™) label-free discovery proteomics workflow. All solvents were HPLC-grade from Sigma-Aldrich and all chemicals where not stated otherwise were obtained from Sigma-Aldrich.

### Sample Preparation

Tissue samples were denatured using Biognosys' Denature Buffer, and reduced and alkylated using Biognosys' Reduction and Alkylation Solution for 60 min at 37° C. Subsequently, digestion to peptides was carried out using trypsin (w/w ratio 1 50 Promega) overnight at 37° C. Samples were prepared using the PreOmics sample preparation kit and frozen as dried peptides. Peptides were resuspended in LC solvent A (1% acetonitrile, 0.1% formicacid (FA)) spiked with Biognosys' iRT kit calibration peptides. Peptide concentrations were determined using a UV/VIS Spectrometer (SPECTROstar Nano, BMG Labtech).

### HPRP Fractionation

Cortex samples (P1, P21, P56): Peptides were pooled according to the genotype (two pools: WT and AS). Ammonium hydroxide was added to both pools to a pH value >10. The fractionation was performed using a Dionex UltiMate 3000RS pump (Thermo Scientific) on an Acquity UPLC CSH C18 1.7 μm, 2.1×150 mm column (Waters). The gradient was 1% to 40% solvent B in 20 minutes, solvents were A: 20 mM ammonium formate in water, B: Acetonitrile. Fractions were taken every 30 seconds and sequentially pooled to 6 fraction pools for mouse cortex samples and 8 for rat brain samples. For mouse cortex samples, the eluates were dried down, resolved in 15 μl solvent A, and spiked with Biognosys' HRM kit calibration peptides, rat samples were resolved in 12 μL solvent A and spiked with Biognosys' iRT kit calibration peptides.

Mouse cortex samples (Ube3a reinstatement): Two pools of peptides were generated (WT; CreERT+ and Ube3aStop/p+; CreERT−, 6 samples each). The two pools were diluted 4× in 0.2 M ammonium formate (pH 10) and applied on C18 MicroSpin columns (The Nest Group). The peptides were then eluted with buffers containing 0.05 M ammonium formate and increasing acetonitrile concentrations (5%, 10%, 15%, 20%, 25%, and 50%) at a pH of 10. The eluates were dried down, resolved in 15 μl solvent A and spiked with Biognosys' HRM kit calibration peptides. Fractions 5% and 50% were pooled. All peptide concentrations were determined using a UV/VIS Spectrometer.

### Shotgun LC MS/MS for Spectral Library Generation

For the LC MS/MS measurements of mouse brain tissues, 2 μg of peptides per fraction (or 4 μg for fraction 5+50% of cortex samples with Ube3a reinstatement) were injected to an in house packed C18 column (Dr. Maisch ReproSil Pur 1.9 μm particle size, 120 Å pore size 75 μm inner diameter, 50 cm length, New Objective) on a Thermo Scientific™ Easy nLC 1200 nano liquid chromatography system connected to a Thermo Scientific Q Exactive™ HF mass spectrometer equipped with a standard nano electrospray source LC solvents were A: 1% acetonitrile in water with 0.1% FA; B: 15% water in acetonitrile with 0.1% FA. The LC gradient was 1-55% solvent B in 60 minutes followed by 55-90% B in 10 seconds, 90% B for 10 minutes, 90%-1% B in 0.1 minutes and 1% B for 5 minutes. A modified TOP15 methods was used [58]

Additionally, mouse Somatosensory cortex 1 barrel field and mouse cerebellum libraries from MCP publication by Bruderer et al. were used for the analysis.

For DDA LC MS/MS measurements of rat brain samples, 1 μg of peptides per fraction were injected to an in-house packed reversed phase column (PicoFrit emitter with 75 μm inner diameter, 60 cm length and 10 μm tip from New Objective, packed with 1.7 μm Charged Surface Hybrid C18 particles from Waters) on a Thermo Scientific EASY-nLC™ 1200 nano-liquid chromatography system connected to a Thermo Scientific Orbitrap Fusion Tribrid mass spectrometer equipped with a Nanospray Flex™ Ion Source. LC solvents were A: 1% acetonitrile in water with 0.1% FA; B: 20% water in acetonitrile with 0.1% FA. The nonlinear LC gradient was 1-59% solvent B in 95 min followed by 59-90% B in 10 seconds, 90% B for 8 min, 90%-1% B in 10 seconds and 1% B for 5 min at 60° C. and a flow rate of 250 nl/min. A modified top speed method (3 s cycle time) from Hebert et al. was used [60].

### Database Search of Shotgun LC MS/MS Data

For developmental time course cortex samples (P1, P21, P56), the mass spectrometric data were analyzed using Biognosys' search engine SpectroMine™, and for cortex samples with Ube3a reinstatement, Biognosys' search engine Pulsar (version 1.0.19846) was used. The false discovery rate on peptide and protein level was set to 1%. A mouse UniProt fasta database (Mus musculus, 2019 Jul. 1) was used for the search engine, allowing for 2 missed cleavages and variable modifications (N-term acetylation, methionine oxidation).

For rat brain tissue samples, mass spectrometric data were analyzed using Biognosys' search engine SpectroMine™ (version 1.0.190808), the false discovery rate on peptide and protein level was set to 1%. A rat UniProt/Trembl. Fasta database (Rattus norvegicus, 2019 Jul. 1) was used for the search engine, allowing for 2 missed cleavages and variable modifications (N-term acetylation, methionine oxidation).

### HRM ID+ Mass Spectrometry Acquisition

For the LC MS/MS HRM measurements, 2 μg of peptides per sample were injected to an in house packed C18 column (Dr. Maisch ReproSil Pur, 1.9 μm particle size, 120 Å pore size; 75 μm inner diameter, 50 cm length, New Objective) on a Thermo Scientific Easy nLC 1200 nano liquid chromatography system connected to a Thermo Scientific Q Exactive HF mass spectrometer equipped with a standard nano electrospray source. LC solvents were A: 1% acetonitrile in water with 0.1% FA; B: 15% water in acetonitrile with 0.1% FA. The nonlinear LC gradient was 1-55% solvent B in 120 minutes followed by 55-90% B in 10 seconds and 90% B for 10 minutes. A DIA method with one full range survey scan and 22 DIA windows was used, the gradient length was 135 min.

### HRM Data Analysis

HRM mass spectrometric data were analyzed using Spectronaut Pulsar software (Biognosys, version 12 and 13.8.190930). The false discovery rate on peptide and protein level was set to 1%, data was filtered using row based extraction. The assay library (protein inventory) generated in this project combined with the ones from MCP (Bruderer et al., 2017) was used for the analysis. The HRM measurements analyzed with Spectronaut were normalized using local regression normalization [61].

### HRM Data Analysis

### Mouse Developmental Time Course

Statistical and Bioinformatics analysis was performed in the R statistical environment [62] and the Perseus software [63].

Protein intensities <20 on the original scale were considered to be below the noise threshold and were marked as missing. Data were log 2-transformed and filtered to contain 50% valid values across all samples. Due to the completeness of DIA data, only 3 proteins were excluded and a total of 7,184 protein groups were retained for further analysis.

Partial Least Squares Discriminant Analysis (PLS-DA) was conducted using the DiscriMiner R Package with default settings and was based on the completely measured 7,029 protein groups.

To identify differentially expressed proteins we employed the same R package [65], which allows for permutation-based false discovery rate control and reports the associated q-value [66]. The Two-Class Unpaired Test with 100 permutations was used to compare AS and WT genotypes at each time point separately. Proteins were classified as statistically significantly differentially expressed, if they met a q-value threshold of 5%.

Pathway analysis was conducted in the Perseus software using the 1D annotation function. Gene Ontology (GO) annotations for each protein were downloaded from UniProt [67]. As multiple enrichment tests were performed, the Benjamini-Hochberg method was used to correct for multiple-hypothesis testing. For completeness pathway enrichment scores are plotted for all 3 comparisons and the significantly regulated ones are marked explicity.

### Rat Brain Regions

A similar analysis strategy as described above was applied to the rat data set. Briefly, protein expression values below 20 were converted to NAs. Data were log 2-transformed and filtered to retain protein groups for which values were measured in at least 50% of all samples, resulting in 7,524 protein groups. PLS-DA analysis was conducted using proteins with measurements across all samples (7,346) using the DiscriMiner R package [64]. Differentially expressed proteins were identified between KO and WT rat samples across each brain region separately. The same function for Two Class Unpaired Test was used and q-value of 0.05 was considered to mark proteins as statistically significantly differentially expressed.

### Mouse Ube3a Reinstatement

Protein expression values below 20 were considered below the detection limit and converted to NAs and data were log 2-transformed. 5,325 protein groups contained measured values in at least 50% of all samples and were used for the subsequent statistical analysis. PLS-DA analysis was conducted on proteins measured across all samples (5,314) using the DiscriMiner R package [64].

The same function with response type set to ‘multiclass’ from the package was used to identify differentially expressed protein groups across 4 conditions: WT, KO, Rescue at p21 and Rescue at p56. To determine which pairwise comparisons were significant the Tukey's Honestly Significant Difference test from the R stats package was used and the alpha level was set to 5%.

To identify up- or down-regulated pathways, the 1D annotation test in the Perseus software was used. For each pairwise comparison KO vs WT, Rescue p21 vs KO, and Rescue p56 vs KO, proteins were first mapped to GO annotations and keywords downloaded from the Uniprot database. Subsequently, enrichment scores were calculated and multiple hypothesis testing correction using the Benjamini-Hochberg procedure applied to identify statistically significant differences. For completeness, pathway enrichment scores were always plotted for all 3 comparisons and the significantly regulated ones are marked explicitly.

### Capillary Western Blot

Protein expression of putative Ube3a targets in mouse brain and hiPSC lysates was analysed by automated capillary western blotting (Sally Sue, Protein Simple). All experimental steps were carried out according to the manufacturer's instructions. Briefly, after protein extraction and quantification, a final sample concentration of 0.25 mg/ml was loaded to the capillary cartridges (12-230 kDa Peggy Sue or Sally Sue Separation Module, #SM-S001). Chemiluminescent protein detection was performed using the Anti Rabbit and Anti Mouse Detection Modules (#DM-001 and #DM-002). The analysis of relative protein expression was carried out with the Compass for SW software (Version 4.1.0, Protein Simple) and statistical analysis with GraphPad Prism Software (Version 8) using ANOVA followed by Tukey's post hoc test.

hIPSC Culture

Cell culture of NPCs and neurons was performed as described in Costa et al. and Pandya et al [27, 68].

### IPSC Stainings and Imaging

Control and AS deletion neurons were cultured in BGAA media. Media was changed one day before treatment. hiPSC derived neurons were treated with 1 UM ASO in PBS for 6 weeks during neuronal differentiation. Post treatment, cells were immediately fixed and stained for TKT (Sigma-Aldrich, HPA029480; 1:200), UBE3a (Sigma-Aldrich, SAB1404508; 1:200) and Map2 (Abcam, ab5392; 1:500) and DAPI as previously described [27].

### Imaging and Imaging Data Analysis

Images were acquired on a Leica TCS SP5 confocal microscope. Z-stack projections were performed using the ‘sum of slices’ command on FIJI (ImageJ). Automated quantification of nuclear UBE3A and TKT expression was performed by using DAPI staining as a mask and measuring the integrated density of fluorescence in each cell. Manual thresholding of HuC/D staining in each independent experiment was performed to distinguish neuronal from non-neuronal cells. Replicates from two independent differentiations were combined for data analysis. Statistical analysis was performed with GraphPad Prism Software (version 8) using Kruskal-Wallis test followed by Dunn's post hoc test.

### ASO Treatments

| | | | Seq. |
|---|---|---|---|
| | | | Id. |
| ASO name | Target | Sequence | No. |
| UBE3A | UBE3A mRNA | TTTAcacctacttcttaaCA | 9 |
| NT | NA | TTGaataagtggaTGT | 10 |

### Immunohistochemistry

Brain tissue was fixed with 4% PFA and prepared for cryosectioning. Immunohistochemical staining for Tkt (Sigma-Aldrich, HPA029480; 1:200), Ube3a (Sigma-Aldrich, SAB1404508; 1:400), and NeuN (Sigma-Aldrich, MAB377; 1/500) in rat brain tissue was performed as previously described [13]. Briefly, sections were blocked with PBS containing 0.5% Triton X-100 and 5% normal horse serum for 1 h at room temperature. Primary antibody labeling was performed in PBS containing 0.5% Triton X-100 and 1% normal horse serum overnight at room temperature. Following primary antibody labeling, sections were washed with PBS and incubated with corresponding Alexa-conjugated secondary antibodies and DAPI in PBS buffer containing 0.5% Triton X-100, 1% normal horse serum for 2 h at room temperature.

### Imaging and Data Analysis

Images were acquired on a Leica TCS SP8 confocal microscope. Z-stack projections were performed using the ‘sum of slices’ command on FIJI (ImageJ). Automated quantification of nuclear UBE3A and TKT expression was performed by using DAPI staining as a mask and measuring the integrated density of fluorescence in each cell. Manual thresholding of NeuN staining was performed and kept consistent across animals to distinguish neuronal from non-neuronal cells. Statistical analysis was performed with GraphPad Prism Software (version 8) using Kruskal-Wallis test followed by Dunn's post hoc test.

Biomarker proteins (human proteins listed) of the present invention.

| | | Uniprot | Seq. |
|---|---|---|---|
| Protein name | Gene name | ID | Id. No. |
| Transketolase | TKT | P29401 | 1 |
| DZANK1 Double zinc ribbon | DZANK1 | Q9NVP4 | 2 |
| and ankyrin repeat-containing | | | |
| protein 1 | | | |
| Acylphosphatase-1 | ACYP1 | P07311 | 3 |
| Ubiquitin-like domain- | UBLCP1 | Q8WVY7 | 4 |
| containing CTD phosphatase 1 | | | |
| Tyrosine--tRNA ligase, cyto- | YARS | P54577 | 5 |
| plasmic | | | |
| Tryptophan--tRNA ligase, | WARS | P23381 | 6 |
| cytoplasmic | | | |
| Superoxide dismutase [Mn], | SOD2 | P09671 | 7 |
| mitochondrial | | | |
| Proteasome activator complex | PSME3 | P61289 | 8 |
| subunit 3 | | | |

### REFERENCES

1. Buiting K, Williams C, Horsthemke B. Angelman syndrome—insights into a rare neurogenetic disorder. Nat Rev Neurol. 2016; 12:584-593.
2. Mabb A M, Judson M C, Zylka M J, Philpot B D. Angelman syndrome: Insights into genomic imprinting and neurodevelopmental phenotypes. Trends Neurosci. 2011; 34:293-303.
3. Rotaru D C, Mientjes E J, Elgersma Y. Angelman Syndrome: From Mouse Models to Therapy. Neuroscience. 2020.
4. Silva-santos S, Woerden G M Van, Bruinsma C F, Mientjes E. Ube3a reinstatement identifies distinct treatment windows in Angelman syndrome model mice. J Clin Invest. 2015; 125:2069-2076.
5. Gu B, Carstens K E, Judson M C, Dalton K A, Rougié M, Clark E P, et al. Ube3a reinstatement mitigates epileptogenesis in Angelman syndrome model mice. J Clin Invest. 2019. 2019. https://doi.org/10.1172/JCI120816.
6. Huang H S, Allen J A, Mabb A M, King I F, Miriyala J, Taylor-Blake B, et al. Topoisomerase inhibitors unsilence the dormant allele of Ube3a in neurons. Nature. 2012. 2012. https://doi.org/10.1038/nature10726.
7. Meng L, Ward A J, Chun S, Bennett C F, Beaudet A L, Rigo F. Towards a therapy for Angelman syndrome by targeting a long non-coding RNA. Nature. 2014; 518:409-412.
8. Meng L, Person R E, Huang W, Zhu P J, Costa-Mattioli M, Beaudet A L. Truncation of Ube3a-ATS Unsilences Paternal Ube3a and Ameliorates Behavioral Defects in the Angelman Syndrome Mouse Model. PLOS Genet. 2013; 9.
9. Elgersma Y, Sonzogni M. UBE3A reinstatement as a disease-modifying therapy for Angelman syndrome. Dev Med Child Neurol. 2021.
10. Egawa K, Kitagawa K, Inoue K, Takayama M, Takayama C, Saitoh S, et al. Decreased tonic inhibition in cerebellar granule cells causes motor dysfunction in a mouse model of angelman syndrome. Sci Transl Med. 2012. 2012. https://doi.org/10.1126/scitranslmed.3004655.
11. Sun J, Zhu G, Liu Y, Standley S, Ji A, Tunuguntla R, et al. UBE3A Regulates Synaptic Plasticity and Learning and Memory by Controlling SK2 Channel Endocytosis. Cell Rep. 2015; 12:449-461.
12. Sun J, Liu Y, Jia Y, Hao X, Lin W J, Tran J, et al. UBE3A-mediated p18/LAMTOR1 ubiquitination and degradation regulate mTORC1 activity and synaptic plasticity. Elife. 2018. 2018. https://doi.org/10.7554/eLife.37993.
13. Avagliano Trezza R, Sonzogni M, Bossuyt S N V, Zampeta F I, Punt A M, van den Berg M, et al. Loss of nuclear UBE3A causes electrophysiological and behavioral deficits in mice and is associated with Angelman syndrome. Nat Neurosci. 2019. 2019. https://doi.org/10.1038/s41593-019-0425-0.
14. Avagliano Trezza R, Punt A M, Mientjes E, van den Berg M, Zampeta F I, de Graaf I J, et al. Mono-ubiquitination of Rabphilin 3A by UBE3A serves a non-degradative function. Sci Rep. 2021. 2021. https://doi.org/10.1038/s41598-021-82319-9.
15. Bossuyt S N V, Punt A M, de Graaf I J, van den Burg J, Williams M G, Heussler H, et al. Loss of nuclear UBE3A activity is the predominant cause of Angelman syndrome in individuals carrying UBE3A missense mutations. Hum Mol Genet. 2021. 2021. https://doi.org/10.1093/hmg/ddab050.
16. Tomaić V, Banks L. Angelman syndrome-associated ubiquitin ligase UBE3A/E6A P mutants interfere with the proteolytic activity of the proteasome. Cell Death Dis. 2015. 2015. https://doi.org/10.1038/cddis.2014.572.
17. Koopmans F, Pandya N J, Franke S K, Phillippens IHCMH, Paliukhovich I, Li K W, et al. Comparative Hippocampal Synaptic Proteomes of Rodents and Primates: Differences in Neuroplasticity-Related Proteins. Front Mol Neurosci. 2018. 2018. https://doi.org/10.3389/fnmol.2018.00364.
18. Ludwig C, Gillet L, Rosenberger G, Amon S, Collins B C, Aebersold R. Data-independent acquisition-based SWATH-M S for quantitative proteomics: a tutorial. Mol Syst Biol. 2018. 2018. https://doi.org/10.15252/msb.20178126.
19. Judson M C, Sosa-Pagan J O, Del Cid W A, Han J E, Philpot B D. Allelic specificity of Ube3a expression in the mouse brain during postnatal Development. J Comp Neurol. 2014. 2014. https://doi.org/10.1002/cne.23507.
20. Sonzogni M, Hakonen J, Bernabé Kleijn M, Silva-Santos S, Judson M C, Philpot B D, et al. Delayed loss of UBE3A reduces the expression of Angelman syndrome-associated phenotypes. Mol Autism. 2019. 2019. https://doi.org/10.1186/s13229-019-0277-1.
21. Kwon N H, Fox P L, Kim S. Aminoacyl-tRNA synthetases as therapeutic targets. Nat Rev Drug Discov. 2019.
22. Tanaka K. The proteasome: Overview of structure and functions. Proc Japan Acad Ser B Phys Biol Sci. 2009.
23. Koopmans F, van Nierop P, Andres-Alonso M, Byrnes A, Cijsouw T, Coba M P, et al. SynGO: An Evidence-Based, Expert-Curated Knowledge Base for the Synapse. Neuron. 2019. 2019. https://doi.org/10.1016/j.neuron.2019.05.002.
24. Berg E L, Pride M C, Petkova S P, Lee R D, Copping N A, Shen Y, et al. Translational outcomes in a full gene deletion of ubiquitin protein ligase E3A rat model of Angelman syndrome. Transl Psychiatry. 2020. 2020. https://doi.org/10.1038/s41398-020-0720-2.
25. Wang T, van Woerden G M, Elgersma Y, Borst J G G. Enhanced transmission at the calyx of held synapse in a mouse model for angelman syndrome. Front Cell Neurosci. 2018. 2018. https://doi.org/10.3389/fncel.2017.00418.
26. Bruinsma C F, Schonewille M, Gao Z, Aronica E M A, Judson M C, Philpot B D, et al. Dissociation of locomotor and cerebellar deficits in a murine Angelman syndrome model. J Clin Invest. 2015. 2015. https://doi.org/10.1172/JCI83541.
27. Pandya N J. Secreted retrovirus-like GAG domain-containing protein PEG10 is regulated by UBE3A and is involved in Angelman syndrome pathophysiology. Cell Reports Med. 2021. 2021. https://doi.org/https://doi.org/10.1016/j.xcrm.2021.100360.
28. Qin Z, Xiang C, Zhong F, Liu Y, Dong Q, Li K, et al. Transketolase (TKT) activity and nuclear localization promote hepatocellular carcinoma in a metabolic and a non-metabolic manner. J Exp Clin Cancer Res. 2019. 2019. https://doi.org/10.1186/s13046-019-1131-1.
29. Li M, Zhang X, Lu Y, Meng S, Quan H, Hou P, et al. The nuclear translocation of transketolase inhibits the farnesoid receptor expression by promoting the binding of HDAC3 to FXR promoter in hepatocellular carcinoma cell lines. Cell Death Dis. 2020. 2020. https://doi.org/10.1038/s41419-020-2225-6.
30. Geerts-Haages A, Bossuyt S N V, den Besten I, Bruggenwirth H, van der Burgt I, Yntema H G, et al. A novel UBE3A sequence variant identified in eight related individuals with neurodevelopmental delay, results in a phenotype which does not match the clinical criteria of Angelman syndrome. Mol Genet Genomic Med. 2020. 2020. https://doi.org/10.1002/mgg3.1481.
31. Zaaroor-Regev D, De Bie P, Scheffner M, Noy T, Shemer R, Heled M, et al. Regulation of the polycomb protein Ring1B by self-ubiquitination or by E6-A P may have implications to the pathogenesis of Angelman syndrome. Proc Natl Acad Sci USA. 2010. 2010. https://doi.org/10.1073/pnas. 1003108107.
32. Zampeta F I, Sonzogni M, Niggl E, Lendemeijer B, Smeenk H, De Vrij F M S, et al. Conserved UBE3A subcellular distribution between human and mice is facilitated by non-homologous isoforms. Hum Mol Genet. 2020. 2020. https://doi.org/10.1093/hmg/ddaa194.
33. Sirois C L, Bloom J E, Fink J J, Gorka D, Keller S, Germain N D, et al. Abundance and localization of human UBE3A protein isoforms. Hum Mol Genet. 2020. 2020. https://doi.org/10.1093/hmg/ddaa191.
34. Yi J J, Paranjape S R, Walker M P, Choudhury R, Wolter J M, Fragola G, et al. The autism-linked UBE3A T485A mutant E3 ubiquitin ligase activates the Wnt/B-catenin pathway by inhibiting the proteasome. J Biol Chem. 2017. 2017. https://doi.org/10.1074/jbc.M117.788448.
35. Martinez-Noel G, Galligan J T, Sowa M E, Arndt V, Overton T M, Harper J W, et al. Identification and Proteomic Analysis of Distinct UBE3A/E6A P Protein Complexes. Mol Cell Biol. 2012. 2012. https://doi.org/10.1128/mcb.00201-12.
36. Jacobson A D, MacFadden A, Wu Z, Peng J, Liu C W. Autoregulation of the 26S proteasome by in situ ubiquitination. Mol Biol Cell. 2014. 2014. https://doi.org/10.1091/mbc.E13-10-0585.
37. Martínez-Noël G, Luck K, Kühnle S, Desbuleux A, Szajner P, Galligan J T, et al. Network Analysis of UBE3A/E6AP-Associated Proteins Provides Connections to Several Distinct Cellular Processes. J Mol Biol. 2018. 2018. https://doi.org/10.1016/j.jmb.2018.01.021.
38. Guo M, Yang X L, Schimmel P. New functions of aminoacyl-tRNA synthetases beyond translation. Nat Rev Mol Cell Biol. 2010.
39. Boczonadi V, Jennings M J, Horvath R. The role of tRNA synthetases in neurological and neuromuscular disorders. FEBS Lett. 2018.
40. Ognjenović J, Simonović M. Human aminoacyl-tRNA synthetases in diseases of the nervous system. RNA Biol. 2018.
41. Farook M F, DeCuypere M, Hyland K, Takumi T, LeDoux M S, Reiter L T. Altered serotonin, dopamine and norepinepherine levels in 15q duplication and Angelman syndrome mouse models. PLOS One. 2012. 2012. https://doi.org/10.1371/journal.pone.0043030.
42. Smirnova E V., Lakunina V A, Tarassov I, Krasheninnikov I A, Kamenski P A. Noncanonical functions of aminoacyl-tRNA synthetases. Biochem. 2012.
43. Maranga C, Fernandes T G, Bekman E, da Rocha S T. Angelman syndrome: a journey through the brain. FEBS J. 2020.
44. Gong X, Tan M, Gao Y, Chen K, Guo G. CRMP-5 interacts with actin to regulate neurite outgrowth. Mol Med Rep. 2016. 2016. https://doi.org/10.3892/mmr.2015.4662.
45. Yamashita N, Mosinger B, Roy A, Miyazaki M, Ugajin K, Nakamura F, et al. CRMP5 (Collapsin Response Mediator Protein 5) regulates dendritic development and synaptic plasticity in the cerebellar purkinje cells. J Neurosci. 2011. 2011. https://doi.org/10.1523/JNEUROSCI.5337-10.2011.
46. Yu Z, Kryzer T J, Griesmann G E, Kim K K, Benarroch E E, Lennon V A. CRMP-5 neuronal autoantibody: Marker of lung cancer and thymoma-related autoimmunity. Ann Neurol. 2001. 2001. https://doi.org/10.1002/1531-8249 (20010201) 49: 2<146::AID-ANA34>3.0.CO; 2-E.
47. Qin L, Liu X, Liu S, Liu Y, Yang Y, Yang H, et al. Differentially expressed proteins underlying childhood cortical dysplasia with epilepsy identified by iTRAQ proteomic profiling. PLOS One. 2017. 2017. https://doi.org/10.1371/journal.pone.0172214.
48. Yeh E, Dao D Q, Wu Z Y, Kandalam S M, Camacho F M, Tom C, et al. Patient-derived iPSCs show premature neural differentiation and neuron type-specific phenotypes relevant to neurodevelopment. Mol Psychiatry. 2018. 2018. https://doi.org/10.1038/mp.2017.238.
49. Schleinitz D, Seidel A, Stassart R, Klammt J, Hirrlinger P G, Winkler U, et al. Novel mutations in the asparagine synthetase gene (ASNS) associated with microcephaly. Front Genet. 2018. 2018. https://doi.org/10.3389/fgene.2018.00245.
50. Sonzogni M, Zhai P, Mientjes E J, Van Woerden G M, Elgersma Y. Assessing the requirements of prenatal UBE3 A expression for rescue of behavioral phenotypes in a mouse model for Angelman syndrome. Mol Autism. 2020. 2020. https://doi.org/10.1186/s13229-020-00376-9.
51. Rotaru D C, van Woerden G M, Wallaard I, Elgersma Y. Adult Ube3a gene reinstatement restores the electrophysiological deficits of prefrontal cortex layer 5 neurons in a mouse model of angelman syndrome. J Neurosci. 2018. 2018. https://doi.org/10.1523/JNEUROSCI.0083-18.2018.
52. Park A J, Havekes R, Fu X, Hansen R, Tudor J C, Peixoto L, et al. Learning induces the translin/trax RNase complex to express activin receptors for persistent memory. Elife. 2017. 2017. https://doi.org/10.7554/eLife.27872.
53. Südhof T C. Synaptic Vesicles: An Organelle Comes of Age. Cell. 2006.
54. Alexander-Kaufman K, Harper C. Transketolase: observations in alcohol-related brain damage research. Int J Biochem Cell Biol. 2009; 41:717-720.
55. Boyle L, Wamelink MMC, Salomons G S, Roos B, Pop A, Dauber A, et al. Mutations in TKT Are the Cause of a Syndrome Including Short Stature, Developmental Delay, and Congenital Heart Defects. Am J Hum Genet. 2016. 2016. https://doi.org/10.1016/j.ajhg.2016.03.030.
56. Kruger N J, Von Schaewen A. The oxidative pentose phosphate pathway: Structure and organisation. Curr Opin Plant Biol. 2003.
57. Snaebjornsson M T, Schulze A. Non-canonical functions of enzymes facilitate cross-talk between cell metabolic and regulatory pathways. Exp Mol Med. 2018.
58. Scheltema R A, Hauschild J-P, Lange O, Hornburg D, Denisov E, Damoc E, et al. The Q Exactive H F, a Benchtop Mass Spectrometer with a Pre-filter, High-performance Quadrupole and an Ultra-high-field Orbitrap Analyzer. Mol Cell Proteomics. 2014; 13:3698-3708.
59. Bruderer R, Bernhardt O M, Gandhi T, Xuan Y, Sondermann J, Schmidt M. Optimization of Experimental Parameters in Data-Independent Mass Spectrometry Significantly Increases Depth and Reproducibility of Results. Mol Cell Proteomics. 2017; 41.
60. Hebert A S, Richards A L, Bailey D J, Ulbrich A, Coughlin E E, Westphall M S, et al. The one hour yeast proteome. Mol Cell Proteomics. 2014. 2014. https://doi.org/10.1074/mcp.M113.034769.
61. Callister S J, Barry R C, Adkins J N, Johnson E T, Qian W J, Webb-Robertson B J M, et al. Normalization approaches for removing systematic biases associated with mass spectrometry and label-free proteomics. J Proteome Res. 2006. 2006. https://doi.org/10.1021/pr0503001.
62. R Core Team. R Core Team (2014). R: A language and environment for statistical computing. R Found Stat Comput Vienna, Austria URL Http//WwwR-ProjectOrg/. 2014. 2014.
63. Tyanova S, Temu T, Sinitcyn P, Carlson A, Hein M Y, Geiger T, et al. The Perseus computational platform for comprehensive analysis of (prote) omics data. Nat Methods. 2016.

64 Gaston A, Sanchez M G. DiscriMiner: Tools of the Trade for Discriminant Analysis. R Packag Version 01-29. 2013. 2013.
65. Rédei G P. SAM (significance analysis of microarrays). Encycl. Genet. Genomics, Proteomics Informatics, 2008.
66. Storey J D. A direct approach to false discovery rates. J R Stat Soc Ser B Stat Methodol. 2002. 2002. https://doi.org/10.1111/1467-9868.00346.
67. Bateman A. UniProt: A worldwide hub of protein knowledge. Nucleic Acids Res. 2019. 2019. https://doi.org/10.1093/nar/gky 1049.
68. Costa V, Aigner S, Vukcevic M, Sauter E, Behr K, Ebeling M, et al. MTORC1 Inhibition Corrects Neurodevelopmental and Synaptic Alterations in a Human Stem Cell Model of Tuberous Sclerosis. Cell Rep. 2016; 15:86-95.

1. A method for measuring UBE3A protein expression modulation in a tissue sample comprising the steps:
a) providing a tissue sample of an animal or cell culture which has been treated with a UBE3A modulator,
b) measuring a protein expression level in the sample of step a) of at least one protein selected from the group consisting of: TKT, DZANK1, ACYP1, UBLCP1, YARS, WARS, SOD2 and PSME3.
c) comparing the protein expression level of the at least one protein measured in step b) to the protein expression level of the at least one protein in a control, wherein a modulated protein expression level of the at least one protein measured in step b) compared to the protein expression level of the at least one protein in the control is indicative for UBE3A protein expression modulation.

2. The method of claim 1 for measuring UBE3A protein expression induction in a tissue sample comprising the steps:
d) providing a tissue sample of an animal or cell culture which has been treated with a UBE3A inducer,
e) measuring a protein expression level in the sample of step a) of at least one protein selected from the group consisting of: TKT, DZANK1, ACYP1, UBLCP1, YARS, WARS, SOD2 and PSME3.
f) comparing the protein expression level of the at least one protein measured in step b) to the protein expression level of the at least one protein in a control, wherein a decreased protein expression level of the at least one protein measured in step b) compared to the protein expression level of the at least one protein in the control is indicative for UBE3A protein expression induction.

3. A method for determining UBE3A target engagement of an UBE3A modulator comprising the steps:
a) providing a tissue sample of an animal or cell culture which has been treated with a UBE3A modulator,
b) measuring a protein expression level in the sample of step a) of at least one protein selected from the group consisting of: TKT, DZANK1, ACYP1, UBLCP1, YARS, WARS, SOD2 and PSME3.
c) comparing the protein expression level of the at least one protein measured in step b) to the protein expression level of the at least one protein in a control, wherein a modulated protein expression level of the at least one protein measured in step b) compared to the protein expression level of the at least one protein in the control is indicative for UBE3A target engagement of the UBE3A modulator.

4. The method of claim 1, wherein the tissue sample is a blood sample, a plasma sample or a CSF sample.

5. The method of claim 1, wherein the protein expression level is measured using Western blotting, Mass spectrometry (MS), Liquid chromatography-mass spectrometry (LC-MS) or Immunoassays.

6. The method of claim 1, wherein the UBE3A modulator is an antisense oligonucleotide, in particular a LNA antisense oligonucleotide.

7. The method of claim 1, wherein the UBE3A modulator is an UBE3A protein expression level inducer for the treatment of Autism Spectrum Disorder, Angelman Syndrome or 15qdup syndrome.

8. The method of claim 1, wherein the protein in step b) is selected from the group consisting of TKT, DZANK1, UBLCP1 and PSME3 and the expression level of these proteins inversely correlates to the UBE3A expression level.

9. The method of claim 1, wherein the protein in step b) is selected from the group consisting of ACYP1, YARS, WARS and SOD2 and the expression level of these proteins directly correlates to the expression level of UBE3A protein expression level.

10. A screening method for the identification of UBE3A protein expression modulators comprising the steps:
a) providing a tissue sample of an animal or cell culture which has been treated with a test compound,
b) measuring a protein expression level in the sample of step a) of at least one protein selected from the group consisting of: TKT, DZANK1, ACYP1, UBLCP1, YARS, WARS, SOD2 and PSME3.
c) comparing the protein expression level of the at least one protein measured in step b) to the protein expression level of the at least one protein in a control, wherein a modulated protein expression level of the at least one protein measured in step b) compared to the protein expression level of the at least one protein in the control is indicative for a UBE3A protein expression modulator.

11.-17. (canceled)

18. The method of claim 3, wherein the tissue sample is a blood sample, a plasma sample or a CSF sample.

19. The method of claim 3, wherein the protein expression level is measured using Western blotting, Mass spectrometry (MS), Liquid chromatography-mass spectrometry (LC-MS) or Immunoassays.

20. The method of claim 3, wherein the UBE3A modulator is an antisense oligonucleotide, in particular a LNA antisense oligonucleotide.

21. The method of claim 3, wherein the UBE3A modulator is an UBE3A protein expression level inducer for the treatment of Autism Spectrum Disorder, Angelman Syndrome or 15qdup syndrome.

22. The method of claim 3, wherein the protein in step b) is selected from the group consisting of TKT, DZANK1, UBLCP1 and PSME3 and the expression level of these proteins inversely correlates to the UBE3A expression level.

23. The method of claim 3, wherein the protein in step b) is selected from the group consisting of ACYP1, YARS, WARS and SOD2 and the expression level of these proteins directly correlates to the expression level of UBE3A protein expression level.


SEQUENCE LISTING (10 sequences)

SEQ ID NO: 1 - protein, 623 aa, Homo sapiens
MESYHKPDQQKLQALKDTANRLRISSIQATTAAGSGHPTSCCSAAEIMAVLFFHTMRYKSQDPRNPHNDRFVLSKGHAAPILYAVWAEAGFLAEAELLNLRKISSDLDGHPVPKQAFTDVATGSLGQGLGAACGMAYTGKYFDKASYRVYCLLGDGELSEGSVWEAMAFASIYKLDNLVAILDINRLGQSDPAPLQHQMDIYQKRCEAFGWHAIIVDGHSVEELCKAFGQAKHQPTAIIAKTFKGRGITGVEDKESWHGKPLPKNMAEQIIQEIYSQIQSKKKILATPPQEDAPSVDIANIRMPSLPSYKVGDKIATRKAYGQALAKLGHASDRIIALDGDTKNSTFSEIFKKEHPDRFIECYIAEQNMVSIAVGCATRNRTVPFCSTFAAFFTRAFDQIRMAAISESNINLCGSHCGVSIGEDGPSQMALEDLAMFRSVPTSTVFYPSDGVATEKAVELAANTKGICFIRTSRPENAIIYNNNEDFQVGQAKVVLKSKDDQVTVIGAGVTLHEALAAAELLKKEKINIRVLDPFTIKPLDRKLILDSARATKGRILTVEDHYYEGGIGEAVSSAVVGEPGITVTHLAVNRVPRSGKPAELLKMFGIDRDAIAQAVRGLITKA

SEQ ID NO: 2 - protein, 752 aa, Homo sapiens
MTAGSVCVPQIIPLRVPQPGKANHEIDNNTLLEMKSDTPDVNIYYTLDGSKPEFLKRIGYGENNTFKYIKPITLPDGKIQVKAIAVSKDCRQSGIVTKVFHVDYEPPNIVSPEDNVENVLKDSSRQEFKNGFVGSKLKKKYKNSENQRSWNVNLRKFPESPLEIPAYGGGSGSRPPTRQSQSPGFAHVSGQKCLTSTEIMRIQRETDFLKCAHCLAPRPSDPFARFCQECGSPVPPIFGCRLPPPEGAQMGLCAECRSLVPMNTPICVVCEAPLALQLQPQASLHLKEKVICRACGTGNPAHLRYCVTCEGALPSSQESMCSGDKAPPPPTQKGGTISCYRCGRWNLWEASFCGWCGAMLGIPAGCSVCPKCGASNHLSARFCGSCGICVKSLVKLSLDRSLALAAEEPRPFSESLNIPLPRSDVGTKRDIGTQTVGLFYPSGKLLAKKEQELASQKQRQEKMSDHKPLLTAISPGRGYWRRQLDHISAHLRCYAQNNPEFRALIAEPRMGKLISATVHEDGCEVSIRLNYSQVSNKNLYLNKAVNFSDHLLSSAAEGDGGLCGSRSSWVSDYSQSTSDTIEKIKRIKNFKTKTFQEKKEQLIPENRLLLKEVGPTGEGRVSVIEQLLDEGADPNCCDEDNRPVITVAVMNKHHEAIPVLVQRGADIDQQWGPLRNTALHEATLLGLAGRESTATLLGCNASIQKKNAGGQTAYDLALNTGDDLVTSLFAAKFGQGLEDQLAQTRSLSLDDC

SEQ ID NO: 3 - protein, 99 aa, Homo sapiens
MAEGNTLISVDYEIFGKVQGVFFRKHTQAEGKKLGLVGWVQNTDRGTVQGQLQGPISKVRHMQEWLETRGSPKSHIDKANFNNEKVILKLDYSDFQIVK

SEQ ID NO: 4 - protein, 318 aa, Homo sapiens
MALPIIVKWGGQEYSVTTLSEDDTVLDLKQFLKTLTGVLPERQKLLGLKVKGKPAENDVKLGALKLKPNTKIMMMGTREESLEDVLGPPPDNDDVVNDFDIEDEVVEVENREENLLKISRRVKEYKVEILNPPREGKKLLVLDVDYTLFDHRSCAETGVELMRPYLHEFLTSAYEDYDIVIWSATNMKWIEAKMKELGVSTNANYKITFMLDSAAMITVHTPRRGLIDVKPLGVIWGKFSEFYSKKNTIMFDDIGRNFLMNPQNGLKIRPFMKAHLNRDKDKELLKLTQYLKEIAKLDDFLDLNHKYWERYLSKKQGQ

SEQ ID NO: 5 - protein, 528 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 6 - protein, 471 aa, Homo sapiens
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 7 - protein, 222 aa, Homo sapiens
MLCRAACSTGRRLGPVAGAAGSRHKHSLPDLPYDYGALEPHINAQIMQLHHSKHHAAYVNNLNATEEKYHEALAKGDVTTQVALQPALKFNGGGHINHTIFWTNLSPKGGGEPKGELLEAIKRDFGSFEKFKEKLTAVSVGVQGSGWGWLGFNKEQGRLQIAACSNQDPLQGTTGLIPLLGIDVWEHAYYLQYKNVRPDYLKAIWNVINWENVTERYTACKK

SEQ ID NO: 8 - protein, 254 aa, Homo sapiens
MASLLKVDQEVKLKVDSFRERITSEAEDLVANFFPKKLLELDSFLKEPILNIHDLTQIHSDMNLPVPDPILLTNSHDGLDGPTYKKRRLDECEEAFQGTKVFVMPNGMLKSNQQLVDIIEKVKPEIRLLIEKCNTVKMWVQLLIPRIEDGNNFGVSIQEETVAELRTVESEAASYLDQISRYYITRAKLVSKIAKYPHVEDYRRTVTEIDEKEYISLRLIISELRNQYVTLHDMILKNIEKIKRPRSSNAETLY

SEQ ID NO: 9 - DNA, 20 bp, synthetic construct
  misc_feature 1..20: note = antisense oligonucleotide
  source 1..20: mol_type = other DNA
tttacacctacttcttaaca

SEQ ID NO: 10 - DNA, 16 bp, synthetic construct
  misc_feature 1..16: note = antisense oligonucleotide
  source 1..16: mol_type = other DNA
ttgaataagtggatgt